(19) United States
(12) Patent Application Publication
(10) Pub. No.: US 2021/0298761 A1
(43) Pub. Date: Sep. 30, 2021

(54) APPLICATOR, MEDICAL DEVICE, AND LOADING METHOD OF CLIP

(71) Applicant: OLYMPUS CORPORATION, Tokyo (JP)

(72) Inventors: Kensuke UESAKA, Hino-shi (JP); Shinya ANSAI, Tachikawa-shi (JP)

(73) Assignee: OLYMPUS CORPORATION, Tokyo (JP)

(21) Appl. No.: 17/344,089

(22) Filed: Jun. 10, 2021

Related U.S. Application Data
(63) Continuation of application No. PCT/JP2018/046136, filed on Dec. 14, 2018.

(51) Int. Cl. A61B 17/128; A61B 17/122

(52) U.S. Cl. CPC A61B 17/1285; A61B 2017/0053; A61B 17/122

(57) ABSTRACT

A medical device includes: a clip; a wire configured to be capable of operating the clip; a link that detachably connects the clip and the wire; a sheath with an inner diameter that can accommodate the link; a handle connected to the sheath; a slider provided on the handle and configured to be movable relative to the handle, the slider being connected to the wire; a first sheath constituting the sheath, the first sheath having a length such that the link is arranged at a position protruding from the first sheath when the slider is advanced with respect to the handle to a maximum extent; and a second sheath constituting the sheath and connected to the first sheath, the second sheath having a length such that the link is arranged at a position accommodated in the sheath when the slider is advanced with respect to the handle to a maximum extent.

### CROSS REFERENCE TO RELATED APPLICATIONS

The present application is a continuation application based on a PCT Patent Application No. PCT/JP2018/046136, filed on Dec. 14, 2018, the entire content of which is hereby incorporated by reference.

### BACKGROUND

### Technical Field

The present disclosure relates to an applicator for treating a target tissue in a body, a medical device (more specifically, a ligation apparatus used to ligate tissue), and a loading method of a clip.

### Background

Conventionally known endoscopic treatment tools are introduced into a patient's body via an endoscopic channel in order to ligate an opening or a blood vessel formed in a target tissue in a body, as described in Japanese Unexamined Patent Application, First Publication No. 2012-500158 (hereinafter referred to as Patent Document 1).

The endoscopic treatment tool described in Patent Document 1 includes a tubular outer sheath, a tubular inner sheath inserted through the outer sheath, a drive wire inserted through the inner sheath, a connecting hook that is connected to the distal end of the drive wire and makes it possible to grasp the clip, a base part that is connected to the proximal end of the inner sheath, and a slider part that is connected to the proximal end of the drive wire and is slidable with respect to the base part. The base part has a fixed base in which two positioning grooves are formed and a rotating base in which one positioning groove is formed.

The endoscopic treatment tool described in Patent Document 1 has three positions, that is, a clip release position where the connecting hook is opened, a clip-fastening position to fasten to the clip grasped by the connecting hook, and a clip-connecting position where the connection hook is closed to grasp the clip and the clip is not fastened.

Specifically, the clip release position is the position where the slider is most pulled out from the base part, the clip-fastening position is the position where the slider is pushed most with respect to the base part, and the clip-connecting position is a position set between the clip release position and the clip-fastening position. In other words, in the endoscopic treatment tool described in Patent Document 1, when the slider is moved to the position closest to the distal end with respect to the base part, the connecting hook protrudes from the distal end of the inner sheath, so as to be opened by its own elastic force.

Generally, a medical device for treating (ligating) a target tissue in the body is configured by connecting a treatment tool (for example, a clip) inserted in the body with an applicator for adjusting the position and orientation of the treatment part at the operator's hand. When treating a target tissue in the body using such a medical device, for example, additional treatment may be performed on a new target tissue that could not be found by a preoperative examination. As an example, when a clip is used as a treatment tool, for example, it is necessary to replace the plurality of clips in order to ligate each of the plurality of target tissues. In other words, in a medical device for treating a target tissue in the body, it is preferable to be able to reload the so-called treatment tool by loading a new treatment tool to the applicator.

On the other hand, when treating one target tissue in the body, it may be necessary to temporarily release the state of grasping the tissue by the treatment tool and then grasp the target tissue again, that is, a so-called re-grasping operation of the treatment tool, for example, when a clip used as a treatment tool is used to accidentally grasp an unintended tissue, or when it is desired to adjust the state of grasping the target tissue.

Therefore, in order to appropriately treat the target tissue in the body, it is preferable that the medical device can reload the treatment portion and the treatment tool can re-grasp the target tissue.

In the endoscopic treatment tool described in Patent Document 1, as described above, when the slider is moved to the clip release position, which is the position where the slider is most pulled out from the base part, the connecting hook is opened. Therefore, in the endoscopic treatment tool described in Patent Document 1, different clips can be loaded to the connecting hooks at the clip release position, and so-called clips can be reloaded.

In the endoscopic treatment tool described in Patent Document 1, when treating the target tissue in the body, in order to adjust the grasping state of the target tissue by the clip, it is necessary to move the slider from the clip connection position to the distal end side with respect to the base part. However, such movement may move the endoscopic treatment tool to the clip open position and release the connection state between the clip and the connection hook. That is, the endoscopic treatment tool described in Patent Document 1 does not consider a re-grasping operation of the target tissue by the treatment tool, and it is not possible to achieve both the reloadability of the treatment tool and re-grasping of the target tissue by the treatment tool.

The present disclosure provides an applicator and a medical device (more specifically, a ligation apparatus) that realize both reloadability of a treatment tool and a re-grasping operation of a target tissue by the treatment tool. Also, the present disclosure provides a loading method of a clip.

### SUMMARY

A medical device includes: a clip; a wire configured to be capable of operating the clip; a link that detachably connects the clip and the wire; a sheath with an inner diameter that can accommodate the link; a handle connected to the sheath; a slider provided on the handle and configured to be movable relative to the handle, the slider being connected to the wire; a first sheath constituting the sheath, the first sheath having a length such that the link is arranged at a position protruding from the first sheath when the slider is advanced with respect to the handle to a maximum extent; and a second sheath constituting the sheath and connected to the first sheath, the second sheath having a length such that the link is arranged at a position accommodated in the sheath when the slider is advanced with respect to the handle to a maximum extent.

The clip may include a first link capable of engaging with the link. The link may be engageable with the first link at a protruding position protruding from the sheath, and disengagement with the first link may be restricted by the sheath at an accommodation position accommodated in the sheath. The sheath may be configured to be mutable such that the sheath can change lengths: a first length, which is a length of the sheath when the link is placed in the accommodation position when the slider is maximally advanced with respect to the handle, and a second length, which is a length of the sheath when the link is placed in the protruding position when the slider is maximally advanced with respect to the handle.

In a state where the first link and the second link are engaged and the length of the sheath is the first length, when the slider is maximally advanced, the connecting portion between the first link and the link may be placed in the sheath.

The medical device may further include: a first arm and a second arm provided on the clip; and a holding tube provided in the clip and formed in a tubular shape, the holding tube being configured so that at least one of the first arm and the second arm can be inserted. The first link may be arranged so as to protrude from the holding tube.

In a state where the first link and the link are engaged and the length of the sheath is the first length, when the slider is maximally advanced, the holding tube and the sheath may be in contact.

The medical device may further include: a connecting member provided on the sheath, the connecting member connecting the first sheath and the second sheath and capable of moving the first sheath to a side of the second sheath side with respect to the second sheath; and a restricting part provided on the sheath and restricting the movement of the first sheath with respect to the second sheath in a state where the sheath has the first length.

The restricting part may restrict a movement of the first sheath with respect to the second sheath by a first force, and the medical device may further include an arm provided on the clip, the arm being configured to be closed by pulling the wire with a second force smaller than the first force.

At least one of the first sheath and the second sheath may be connected to the connecting member in a state of being in a cavity formed in the connecting member.

At least a part of either the first sheath or the second sheath may be configured by a spring. The sheath may be configured to be mutable with a first length, which is a length of the sheath when the link is accommodated in the sheath due to elastic deformation of the spring, and a second length, which is a length of the sheath when the link protrudes from the sheath.

By transitioning between a form in which the first sheath and the second sheath are attached and a form in which the first sheath and the second sheath are separated, the sheath may be configured to be mutable with a first length, which is a length of the sheath when the link is accommodated in the sheath due to elastic deformation of the spring, and a second length, which is a length of the sheath when the link protrudes from the sheath.

An applicator configured to be connectable to a clip, the applicator including: a link capable of being connected to the clip; a wire connected to the link; a sheath that accommodates the link; a handle connected to the sheath; a slider provided on the handle and configured to be movable relative to the handle, the slider being connected to the wire; a first sheath constituting the sheath, the first sheath having a length such that the link is arranged at a position protruding from the first sheath when the slider is maximally advanced with respect to the handle; and a second sheath, which constitutes the sheath and is connected to the first sheath, the second sheath having a length to be arranged in a position where the link is accommodated in the sheath when the slider is maximally advanced with respect to the handle.

The applicator may further include: a connecting member provided on the sheath, the connecting member connecting the first sheath and the second sheath and capable of moving the first sheath to a side of the second sheath with respect to the second sheath; and a restricting part provided on the sheath and restricting the movement of the first sheath with respect to the second sheath in a state where the sheath has a first length.

The restricting part may restrict a movement of the first sheath with respect to the second sheath by a first force, and the first force may be larger than a second force, which is a traction force of a wire required when an arm provided in the clip closes.

At least a part of either the first sheath or the second sheath may be configured by a spring, the sheath may be configured to be mutable or changeable between a first length, which is a length of the sheath when the link is accommodated in the sheath due to elastic deformation of the spring, and a second length, which is a length of the sheath when the link protrudes from the sheath.

By transitioning between a form in which the first sheath and the second sheath are attached and a form in which the first sheath and the second sheath are separated, the sheath may be configured to be mutable or changeable between a first length, which is a length of the sheath when the link is accommodated in the sheath due to elastic deformation of the spring, and a second length, which is a length of the sheath when the link protrudes from the sheath.

A loading method of a clip, includes: changing a length of the sheath of the applicator from a first length, which is a length of the sheath when the link accommodating in the sheath is accommodated in the sheath, to a second length, which is a length of the sheath when the link protrudes from the sheath; and engaging the link with the clip in a state where the sheath is at the second length.

The loading method may further includes: changing the length of the sheath from the first length to the second length by elastically deforming a spring forming a part of the sheath.

The mounting method may further include: deforming the spring elastically by pressing the sheath against a cartridge containing the clip.

According to each of the above-described aspects of the medical device and the applicator, it is possible to realize both the reloadability of the treatment tool and a re-grasping operation of the target tissue by the treatment tool. Further, according to each of the above aspects of the loading method of the clip, the clip can be suitably loaded to the medical device by engaging the clip with the applicator.

### BRIEF DESCRIPTION OF THE DRAWINGS

FIG. 1A is a partial sectional view in plan view schematically showing a configuration of a clip according to a first embodiment of the present invention.

FIG. 1B is a side sectional view schematically showing a configuration of a clip according to the present embodiment.

FIG. 2A is a perspective view schematically showing a configuration of a clip case according to the present embodiment.

FIG. 2B is a partial cross-sectional view of the clip case according to the present embodiment in a side view.

FIG. 3 is a side sectional view schematically showing a configuration of an applicator according to the present embodiment.

FIG. 4 is a diagram showing an operation of loading the clip according to the present embodiment to an applicator.

FIG. 5 is a diagram showing an operation of loading the clip according to the present embodiment to an applicator.

FIG. 6A is a diagram showing an operation of loading the clip according to the present embodiment to an applicator.

FIG. 6B is a perspective view showing a connecting configuration of a treatment tool main body and a clip according to the present embodiment.

FIG. 7 is a diagram showing an operation of loading the clip according to the present embodiment to an applicator.

FIG. 8 is a diagram showing an operation of loading the clip according to the present embodiment to an applicator.

FIG. 9 is a diagram showing an operation of treating a target tissue using the medical device according to the present embodiment.

FIG. 10 is a diagram showing an operation of treating a target tissue using the medical device according to the present embodiment.

FIG. 11 is a diagram showing an operation of treating a target tissue using the medical device according to the present embodiment.

FIG. 12A shows an operation of treating a target tissue using the medical device according to the present embodiment.

FIG. 12B is a diagram showing an operation of treating a target tissue using the medical device according to the present embodiment.

FIG. 13A is a side sectional view showing a configuration of an applicator according to a modified example of the present embodiment.

FIG. 13B is a diagram showing an operation of an applicator according to this modification with respect to a connecting member.

FIG. 13C is a diagram showing an operation of an applicator according to this modification with respect to a connecting member.

FIG. 14A is a perspective view showing a configuration of a part of an applicator according to a second embodiment of the present invention.

FIG. 14B is a perspective view showing a configuration of a part of an applicator according to the present embodiment.

FIG. 14C is a perspective view showing a configuration of a part of an applicator according to the present embodiment.

FIG. 15A is a diagram showing an operation of loading the clip according to the present embodiment to an applicator.

FIG. 15B is a perspective view showing a state in which a clip in the medical device according to the present embodiment is loaded to an applicator.

FIG. 15C is a partial cross-sectional view showing a connection configuration between a clip and an applicator according to the present embodiment.

FIG. 16 is a partial cross-sectional view schematically showing the configuration of an applicator according to a third embodiment of the present invention.

FIG. 17 is a diagram showing an operation of loading the clip according to the present embodiment to an applicator.

FIG. 18A is a diagram showing an operation of loading the clip according to the present embodiment to an applicator.

FIG. 18B is a diagram showing an operation of loading the clip according to the present embodiment to an applicator.

FIG. 18C is a diagram showing an operation of loading the clip according to the present embodiment to an applicator.

FIG. 18D is a diagram showing an operation of loading the clip according to the present embodiment to an applicator.

FIG. 19A shows an operation of treating a target tissue using the medical device according to the present embodiment.

FIG. 19B is a diagram showing an operation of treating a target tissue using the medical device according to the present embodiment.

FIG. 20 is a diagram showing an operation of treating a target tissue using the medical device according to the present embodiment.

### DETAILED DESCRIPTION

### First Embodiment

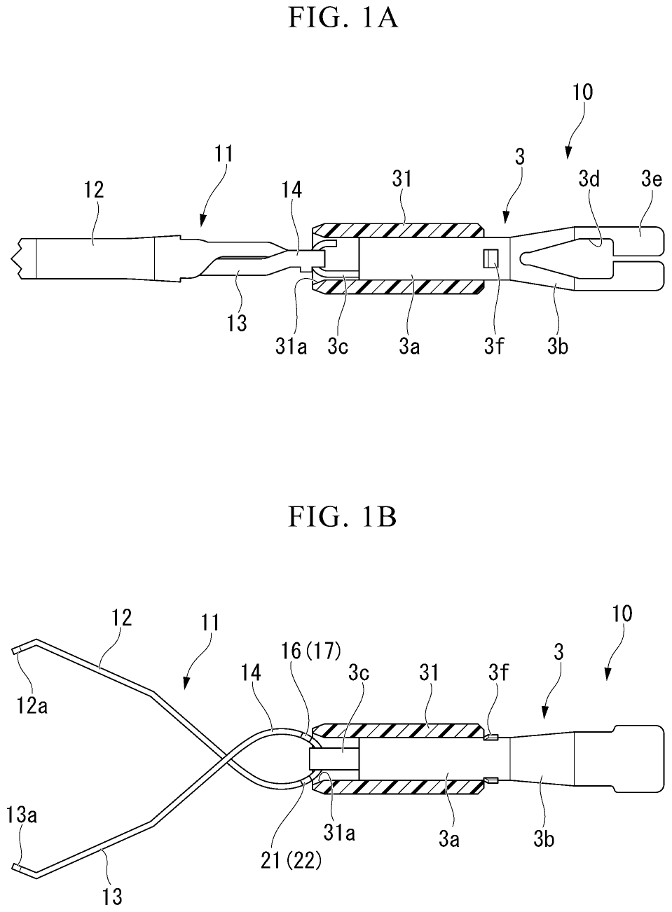

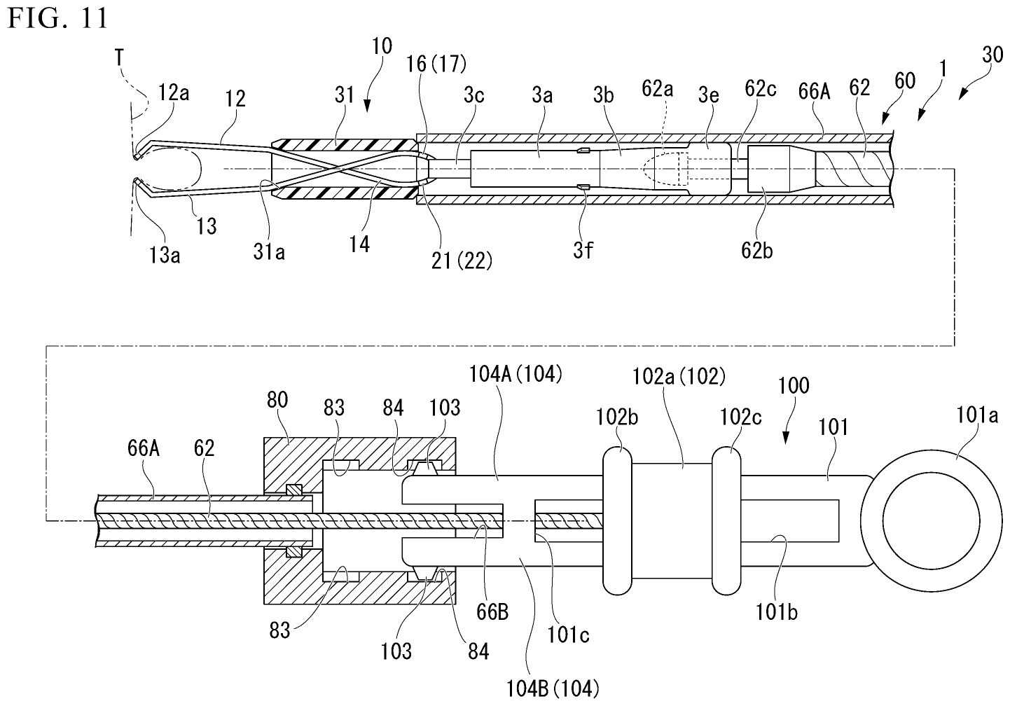

Hereinafter, the configuration of a medical device according to a first embodiment of the present disclosure will be described with reference to FIGS. 1A to 12B.

A medical device 1 according to the present embodiment is used by being inserted into the patient's body through a channel formed in an endoscope (not shown). More specifically, the medical device 1 according to the present embodiment is a ligation apparatus for ligating a target tissue in the body.

In the present specification, the side where the endoscope operating part for the operator to operate the endoscope is positioned is defined as the proximal end side, and the side where the distal end portion of the endoscope inserted into the body is positioned is defined as the distal end side.

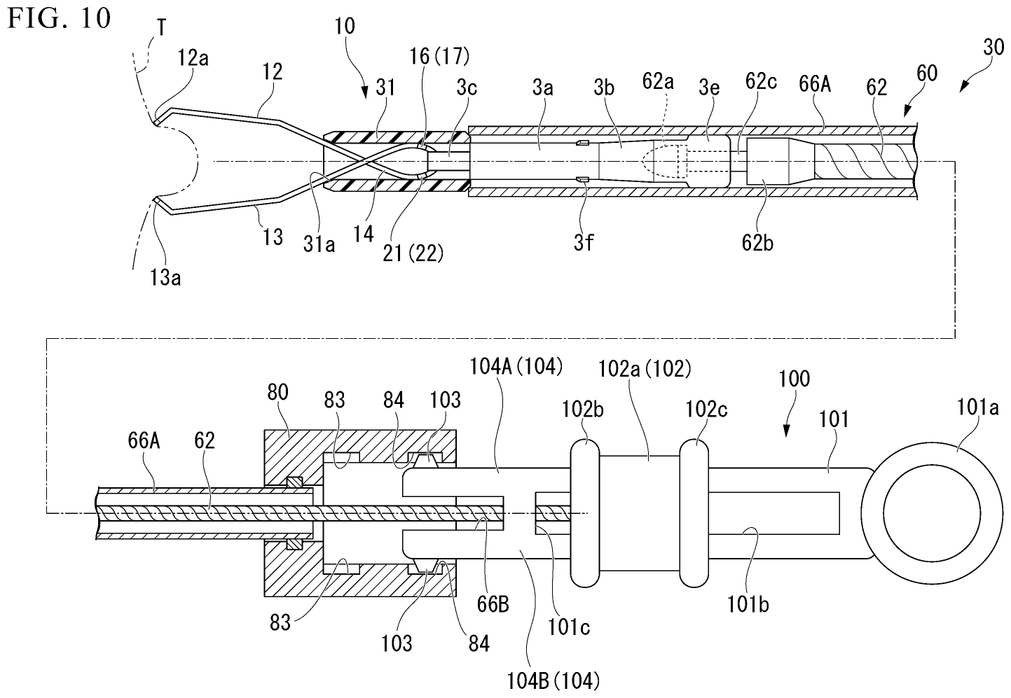

The medical device 1 according to the present embodiment includes a clip (treatment tool) 10 provided on the distal end side and an applicator 30 configured to be connectable to the clip 10 (see FIG. 10). Hereinafter, for convenience of explanation, the clip 10 will be abbreviated as the clip 10.

### (Configuration of Clip)

FIG. 1A is a partial sectional view in plan view schematically showing the configuration of the clip 10 according to the present embodiment. FIG. 1B is a side sectional view of the clip 10.

As shown in FIG. 1A, the clip 10 according to the present embodiment includes an arm member 11, a holding tube 31, and a connecting rod (first link) 3. For convenience of explanation, the longitudinal axis C1 of the insertion portion 60, which will be described later, is regarded as the axis of the clip 10.

### (Configuration of Arm Member)

The arm member 11 has a first arm 12, a second arm 13, and an intermediate portion 14. The first arm 12 and the second arm 13 extend from the proximal end side toward the distal end side and are arranged so as to face each other. As shown in FIG. 1A, the first arm 12 and the second arm 13 may be formed at positions line-symmetrical with respect to the longitudinal axis C1.

In the present embodiment, as shown in FIG. 1A, the intermediate portion 14 is a part formed in the arm member 11 in a loop shape from the portion where the first arm 12 and the second arm 13 intersect to the proximal end side. That is, it can be said that the intermediate portion 14 is located between the proximal end portion of the first arm 12 and the proximal end portion of the second arm 13.

In the present embodiment, the first arm 12 and the second arm 13 are separated from each other in a natural state and have an elastic restoring force so that the distance between them increases along the direction from the proximal end side to the distal end side. In the present specification, the “natural state” means a state in which an external force does not act on the arm member 11. A claw 12a extending toward the second arm 13 side is formed at the distal end of the first arm 12. A claw 13a extending toward the first arm 12 side is formed at the distal end of the second arm 13. The first arm 12 and the second arm 13 of the arm member 11 have an elastic restoring force that moves in the direction in which the distal ends thereof are separated from each other, that is, in the direction in which the arm member 11 opens.

The arm member 11 having the first arm 12 and the second arm 13 can be formed by forming the first arm 12 and the second arm 13 by bending a metal material such as a leaf spring material such as stainless steel and then crossing the first arm 12 and the second arm 13. With this configuration, the arm member 11 can slide along the inner peripheral surface of the holding tube 31 when it is pulled into the holding tube 31 described later.

A pair of protrusions 16 and 17 are formed in the intermediate portion 14 of the arm member 11. More specifically, the pair of protrusions 16 and 17 protrude at right angles to the longitudinal direction in which the first arm 12 and the second arm 13 of the arm member 11 extend. The pair of protrusions 16 and 17 may be formed at positions line-symmetrical with respect to the longitudinal axis C1. By forming a pair of protrusions 16 and 17 of the clip 10, when the arm member 11 is pulled into the holding tube 31, by contacting the inner wall of the holding tube 31, the movement of the arm member 11 with respect to the holding tube 31 can be restricted. In the present embodiment, an example in which the arm member 11 is configured to include the first arm 12 and the second arm 13 which are a pair of arms will be described, but the present invention is not limited thereto. For example, the arm member may be configured by connecting one rod-shaped member and one elastically deformable arm.

### (Configuration of Holding Tube)

In the present embodiment, the holding tube 31 is formed in a cylindrical shape and has an inner diameter through which at least a part of the arm member 11 can enter. That is, the holding tube 31 is formed with a lumen having an inner diameter that allows at least one of the first arm 12 and the second arm 13 of the arm member 11 to enter. In the present embodiment, the holding tube 31 is formed so that the outer diameter is larger than the inner diameter of the sheath (first sheath) 66A of the insertion portion 60 of the applicator 30, which will be described later. Further, a tapered surface 31a is formed on the inner wall of the distal end of the holding tube 31 over the entire circumference. The diameter of the tapered surface 31a increases toward the distal end side.

### (Configuration of Connecting Rod)

As shown in FIGS. 1A and 1B, the connecting rod 3 is formed to have a distal end portion 3a, a proximal end portion 3b, a hook portion 3c, a notch portion 3d, a deformed portion 3e, and a protrusion portion 3f. The connecting rod 3 is formed by a method such as injection molding using a resin material having a predetermined strength.

The connecting rod 3 is configured to connect the arm member 11 and the operation wire 62 described later. In other words, the connecting rod 3 is configured to connect the clip 10 and the applicator 30.

The distal end portion 3a of the connecting rod 3 is configured to support the hook portion 3c for connecting the arm member 11, and the shape thereof is not particularly limited. For example, the distal end portion 3a may be formed in a rod shape having an outer diameter slightly smaller than the inner diameter of the holding tube 31. The distal end portion 3a can move back and forth within the holding tube 31.

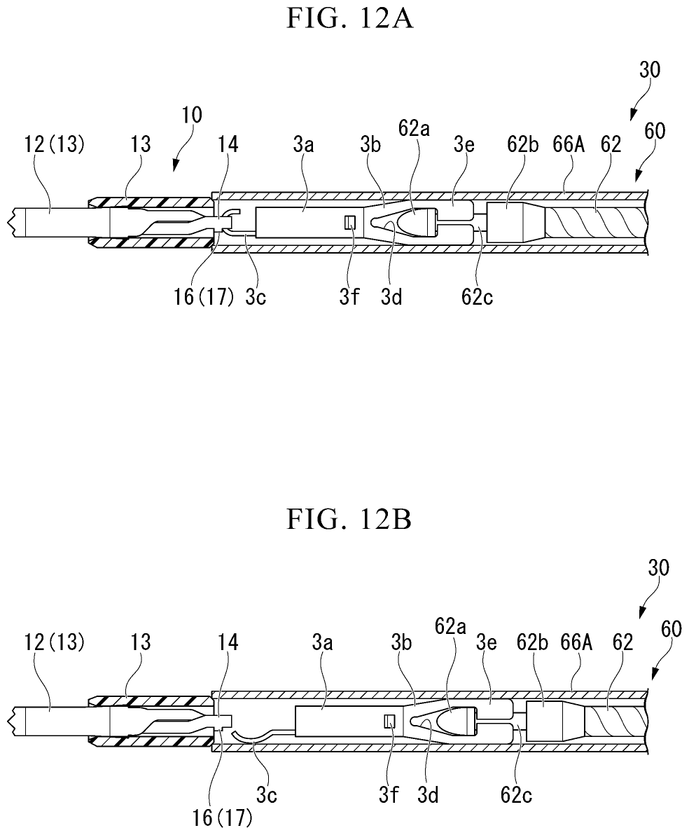

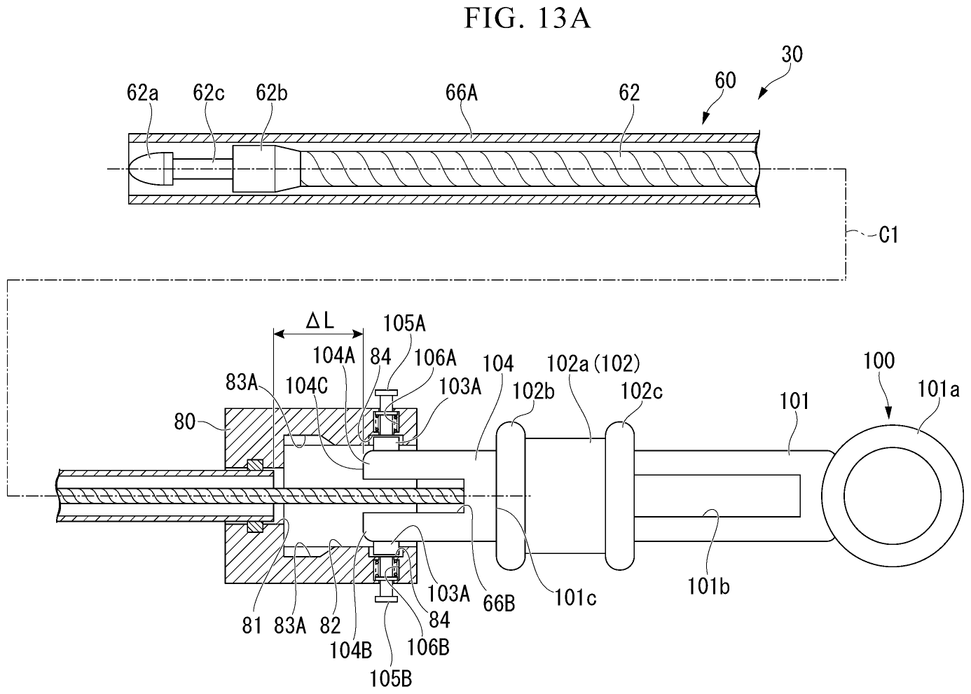

The hook portion 3c is formed so as to protrude in a direction intersecting the longitudinal direction in which the connecting rod 3 extends from the distal end portion 3a. The hook portion 3c can connect the arm member 11 to the connecting rod 3 by hooking the hook portion 3c into a loop shape formed in the intermediate portion 14 of the arm member 11. In the present embodiment, the hook portion 3c is removed from the intermediate portion 14 by bending and deforming on the proximal end side protruding from the distal end portion 3a due to the traction force transmitted to the proximal end side transmitted by the operation wire 62 described later (See FIG. 12B).

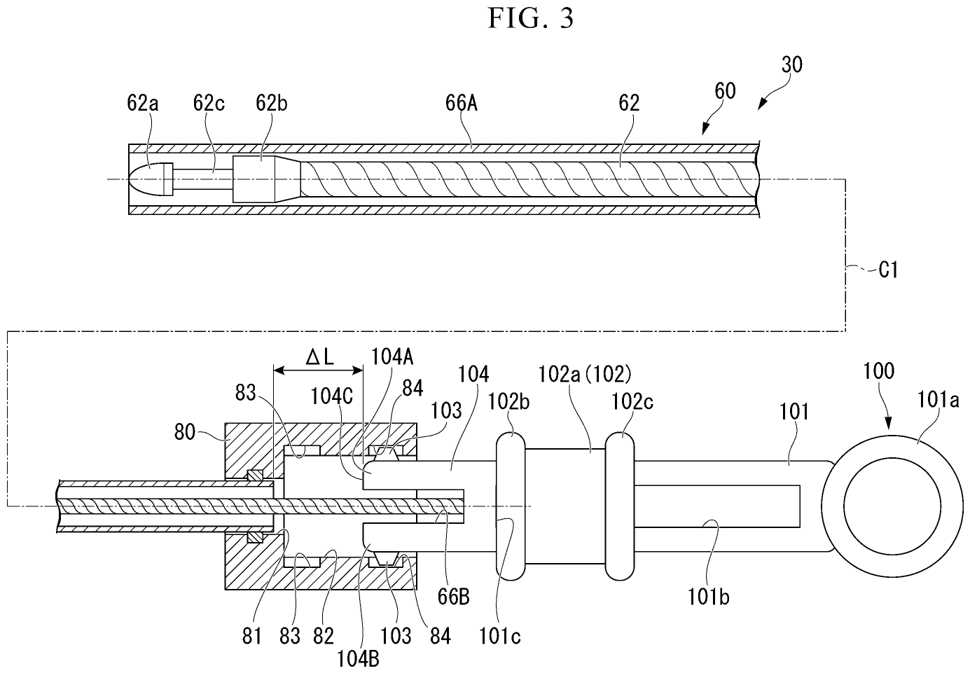

The proximal end portion 3b is formed by being connected to the proximal end side of the distal end portion 3a. In the present embodiment, the notch portion 3d and the deformed portion 3f are formed at the proximal end portion 3b. A notch 3d is formed in the proximal end portion 3b by cutting a part of the proximal end portion 3b according to the shape of the hook 62a (see FIG. 3) provided at the distal end of the operation wire 62 described later. The notch 3d is formed with a size sufficient to completely accept the hook 62a described later.

On the proximal end side of the proximal end portion 3b, the deformed portion 3e is bifurcated to form a pair of tails. As shown in FIG. 1A, a through hole continuous to the notch portion 3d is formed such that the diameter can be expanded to a size that allows the hook 62a, which will be described later, to enter between the pair of tails of the deformed portion 3e by elastic deformation. Although the details will be described later, when the hook 62a is inserted into the through hole formed in the deformed portion 3e, while the outer peripheral surface of the hook 62a is in contact with the inner peripheral surface of the through hole, the pair of tails of the deformed portion 3e are pressed outward in the radial direction of the connecting rod 3. Therefore, each of the pair of tails formed in the deformed portion 3e is elastically deformed outward in the radial direction of the connecting rod 3, and the inner diameter of the through hole is expanded to a size that allows the hook 62a to enter.

A protrusion 3f is formed on the outer peripheral surface of the distal end 3a on the proximal end side where the connecting rod 3 protrudes from the proximal end opening of the holding tube 31. The connecting rod 3 can engage with the holding tube 31 at the position where the protrusion 3f is formed. In the present embodiment, by the elastic restoring force of the first arm 12 and the second arm 13 of the arm member 11, the force for moving the connecting rod 3 toward the distal end side acts on the hook portion 3c of the connecting rod 3. By having the protrusion 3f, the connecting rod 3 can restrict the movement of the connecting rod 3 toward the distal end side with respect to the holding tube 31.

In the present embodiment, as shown in FIGS. 1A and 1B, the connecting rod 3 is arranged in a state where at least the deformed portion 3e of the proximal end portion 3b is located on the proximal end side of the opening on the proximal end side of the holding tube 31. In other words, the connecting rod 3 is arranged in a state in which the deformed portion 3e on the proximal end side always protrudes from the opening on the proximal end side of the holding tube 31.

These members constituting the clip 10, including the arm member 11, are formed of a material such as a cobalt-chromium alloy, titanium, or stainless steel. The clip 10 is configured so that it can be observed under MRI (magnetic resonance imaging) fluoroscopy.

The arm member 11 is formed integrally by punching, for example, a plate material made of a cobalt-chromium alloy or the like into a shape in which the first arm 12, the second arm 13, the intermediate portion 14, and the pair of protrusions 16 and 17 are developed in a plane shape.

The holding tube 31 may be integrally formed of, for example, a material such as a 64 titanium alloy (Ti-6AL-4V) or a cobalt-chromium alloy.

### (Configuration of Clip Cartridge)

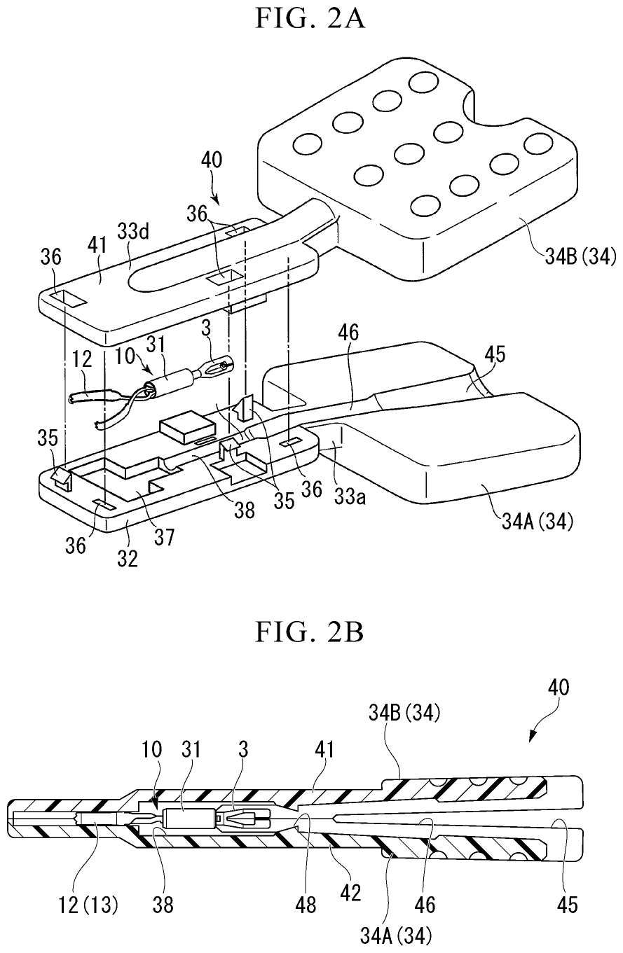

The clip 10 according to the present embodiment is easy to transport in the process of being actually used after being manufactured, and in order to prevent contamination by the external environment, for example, as shown in FIGS. 2A and 2B, the clip 10 is accommodated in the cartridge 40. FIG. 2A is a perspective view schematically showing the configuration of the clip cartridge 40 according to the present embodiment. FIG. 2B is a side sectional view showing a state in which the clip 10 is accommodated in the clip cartridge 40.

As shown in FIG. 2A, the clip cartridge 40 is configured by an upper case 41 having the same shape and the same dimensions, and a lower case 42. The upper case 41 and the lower case 42 can be formed of, for example, various known resin materials having a certain hardness and being transparent. The clip cartridge 40 according to the present embodiment may be formed in a size that can suitably accommodate the clip 10 and is easy for the operator to hold, and the shape is not particularly limited. For example, the clip cartridge 40 may be formed to have a length of about 50 mm, a width of about 10 to 20 mm, and a thickness of about 5 mm.

As shown in FIG. 2A, the upper case 41 and the lower case 42 of the clip cartridge 40 are connected by engaging the three sets of engaging claws 35 and the engaging holes 36 so as to correspond to each other. As shown in FIG. 2A, in a state where the upper case 41 and the lower case 42 of the clip cartridge 40 are connected, the arm member 11 of the clip 10 is accommodated inside the clip storage portion 37 formed in the lower case 42. In this state, the holding tube 31 and the connecting rod 3 of the clip 10 are accommodated in the groove 38 formed on the proximal end side of the clip accommodating portion 37.

In the clip cartridge 40, when the hook 62a, which will be described later, enters the notch 3d through the through hole formed in the proximal end portion 3b of the connecting rod 3, the groove 38 has an inner diameter such that the proximal end portion 3b of the connecting rod 3 can be elastically deformed outward in the radial direction. As shown in FIG. 2B, when the clip 10 is accommodated in the clip storage portion 37, the first arm 12 and the second arm 13 of the arm member may abut on the distal end surface of the clip storage portion 37.

As shown in FIGS. 2A and 2B, a compression portion 34 that can be pressed by the operator is formed on the proximal end side of the clip cartridge 40 with respect to the clip storage portion 37. The compression portion 34 has a pair of compression portions 34A and 34B formed by being bent at a predetermined angle with respect to the direction of the longitudinal axis of the clip cartridge 40 in a state where no external force acts. As shown in FIG. 2B, between the pair of squeezed portions 34A and 34B, a groove 46 and an inlet portion 45 continuously formed in the groove 38 are formed. Although the details will be described later, in the present embodiment, the applicator 30 described later can be inserted into the clip cartridge 40 via the inlet portion 45 and the groove 46.

As described above, the configuration of the lower case 42 of the clip case 40 according to the present embodiment has been described, but since the upper case 41 has the same configuration as the lower case 42, the description thereof will be omitted.

### (Applicator Configuration)

Next, the configuration of the applicator 30 according to the present embodiment will be described with reference to FIG. 3. As shown in FIG. 3, the applicator 30 according to the present embodiment includes an insertion portion 60, an operating portion 100, and a connecting member 80 provided between the insertion portion 60 and the operating portion 100. The insertion portion 60 is located on the distal end side of the operating portion 100, and includes a sheath 66A and an operation wire 62 inserted into the sheath 66A.

### (Configuration of Insertion Part)

The insertion portion 60 includes a sheath 66A and an operation wire 62. The operation wire 62 is inserted into the sheath 66A so as to be able to advance and retreat. The operation wire 62 is configured by connecting the proximal end to the operating portion 100 and connecting the distal end to a hook 62a formed having an arrowhead shape. More specifically, the operation wire 62 may be connected to the concave portion 102 of the slider 102 of the operating portion 100, which will be described later.

The operation wire 62 is provided for the operator to transmit the force for operating the operating portion 100 on the proximal end side (for example, the operation of pushing the slider 102 of the operating portion 100 and the operation of pulling back the slider 102) to the clip 10. In other words, the operation wire 62 is provided for operating the clip 10.

In the present embodiment, the operation wire 62 is formed of, for example, a single metal wire or a stranded wire. As shown in FIG. 3, on the distal end side of the operation wire 62, an integrated configuration is provided in which a hook (second link) 62a formed in an arrowhead shape and a large diameter portion 62b are connected by a shaft portion 62c.

The hook 62a is formed in a conical shape. Specifically, the hook 62a has an outer peripheral surface having a slope shape in which the outer diameter gradually decreases toward the distal end side. The outer diameter of the proximal end surface of the hook 62a is larger than the inner diameter of the through hole of the proximal end portion 3c when the deformed portion 3e of the connecting rod 3 is not elastically deformed. That is, in a state where the hook 62a is accommodated in the notch 3d of the connecting rod 3, the hook 62a does not fall out of the notch 3d only by the advancing/retreating operation of the operation wire 62. The large diameter portion 62b is a cylindrical member made of a metal material such as stainless steel. The distal end of the operation wire 62 is fixed to the large diameter portion 62b by various known methods such as adhesion and welding.

In the present embodiment, the operation wire 62, the hook 62a, the large diameter portion 62b, and the shaft portion 62c are integrally formed. Therefore, the hook 62a can move forward and backward together with the operation wire 62 by moving the operation wire 62 forward and backward.

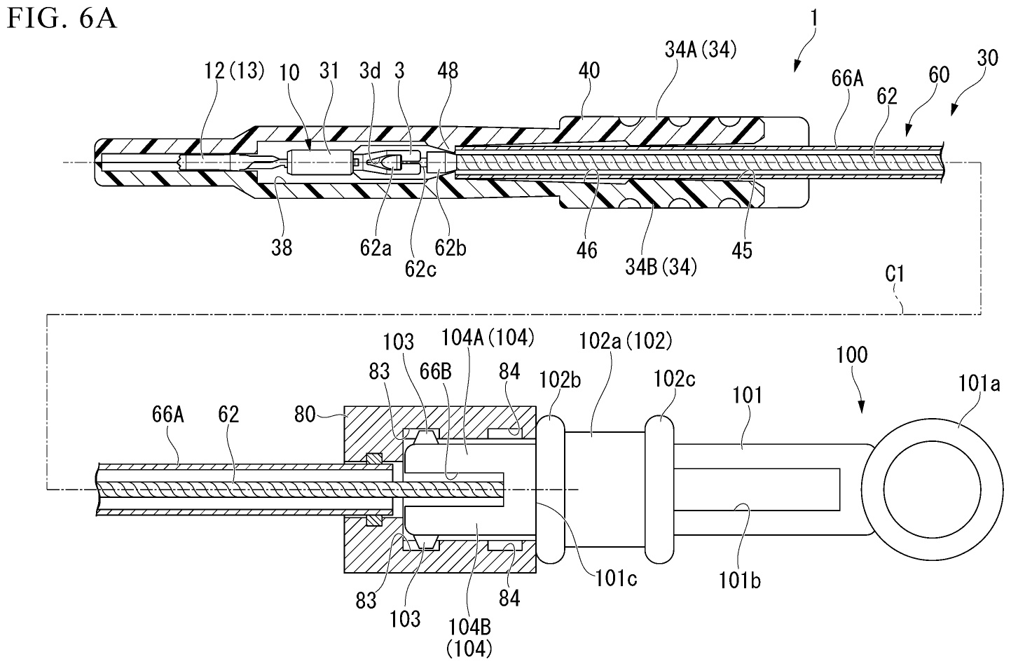

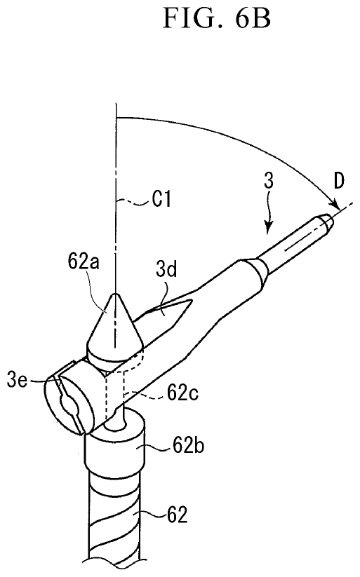

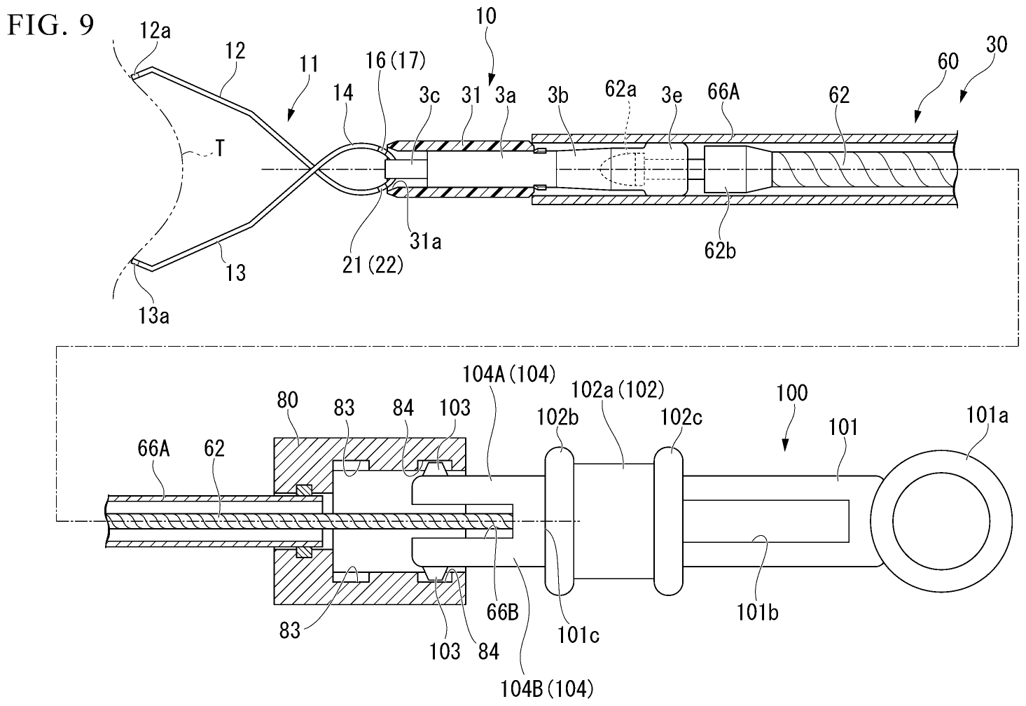

The sheath 66A may be a coil sheath made of stainless steel such as SUS301 having high compression strength, for example. In this case, as the sheath 66A, a coil formed by tightly winding a wire (not shown) in the axial direction C1 can be used. The sheath 66A has flexibility and is strong against compressive force in the axial direction C1. In the present embodiment, when the sheath 66A is located in the sheath 66A in a state where the hook 62a and the notch 3d of the connecting rod 3 are engaged (see FIG. 9), the connecting rod 3 is formed to have an inner diameter that cannot be rotated with respect to the hook 62a. More specifically, as shown in FIG. 6B, in the above-described state, the connecting rod 3 rotates in a direction intersecting the hook 62a in the longitudinal axis C1 direction, and the hook 62a and the connecting rod 3 are engaged with each other. It does not mean that the connecting rod 3 does not rotate with respect to the hook 62a at all.

In the present embodiment, when the hook 62a and the notch 3d of the connecting rod 3 are engaged with each other and are located inside the sheath 66A, the deformation spreading to the outside of the deformed portion 3e of the connecting rod 3 is suppressed by the sheath 66A. In other words, in this state, the deformed portion 3e of the connecting rod 3 is deformed, so that the engagement between the hook 62a and the notch portion 3d is maintained by the sheath 66A even if the width of the connecting rod 3 in the deformed portion 3e is increased.

### (Configuration of Connecting Member)

The connecting member 80 is configured to connect the inserting portion 60 and the operating portion 100. The connecting member 80 has a longitudinal axis and is formed in a cylindrical shape. The connecting member 80 has a first lumen 81 having an inner diameter sufficient to insert the sheath 66A on the distal end side, and a second lumen 82 located on the proximal end side of the first lumen 81 and continuously formed on the first lumen 81. The distal end of the operating portion 100, which will be described later, is inserted into the second lumen 82 so that it can be engaged with the second lumen 82.

On the inner peripheral surface of the second lumen 82, the distal end concave portion 83 and the proximal end concave portion 84 are formed by cutting a part from the inner peripheral surface of the second lumen 82 to the outside in the radial direction of the connecting member 80 at two positions separated by a predetermined distance along the direction of the longitudinal axis of the connecting member 80. As shown in FIG. 3, in the present embodiment, the distal end concave portion 83 and the proximal end concave portion 84 are formed having a rectangular cross-sectional shape. The depth between the distal end concave portion 83 and the proximal end concave portion 84 is not particularly limited, but it suffices that a pair of convex portions 103 formed on the distal end of the operating portion 100, which will be described later, can enter and engage with the distal end concave portion 83 and the proximal end concave portion 84. The distal end concave portion 83 and the proximal end concave portion 84 may be formed over the entire circumference of the inner peripheral surface of the connecting member 80 on which the second lumen 82 is formed, or for example, it may be formed only at two locations according to the pair of convex portions 103 formed at the distal end portion of the operating portion 100.

In the present embodiment, as shown in FIG. 3, the operation wire 62 is connected to the operating portion 100 by inserting the first lumen 81 and the second lumen 82 formed in the connecting member 80. Further, the proximal end portion of the sheath 66A of the insertion portion 60 is inserted into the first lumen 81 of the connecting member 80 and fixed to the inner peripheral surface of the first lumen 81 of the connecting member 80. In other words, the sheath 66A can move forward and backward together with the connecting member 80 by moving forward and backward in the longitudinal axis direction of the connecting member 80.

The present invention is not limited to this. For example, the sheath 66A may be fixed to the connecting member 80 in a state where the proximal end surface is in contact with the distal end surface of the connecting member 80. In this case, the first lumen of the connecting member 80 may have an inner diameter sufficient to allow only the operation wire 62 to be inserted.

As shown in FIG. 3, the distal end portion 104 of the operating portion 100, which will be described later, is inserted into the second lumen 82 through the opening on the proximal end side of the connecting member 80, forming a so-called nested configuration and being engaged with each other. Although the details will be described later, the length L of the sheath 66, which will be described later, can be adjusted by grasping the operating member 80 and moving it toward the proximal end side with respect to the operating portion 100.

In the present embodiment, an example in which the sheath 66A is inserted into and fixed to the connecting member 80 has been described, but the present invention is not limited thereto. For example, the sheath 66B may be fixed in a state of being inserted into the connecting member 80. In this case, the sheath 66A and the connecting member 80 are engaged in a nested configuration. Therefore, the length L of the sheath 66 can be adjusted in the same manner by grasping the operating member 80 and moving it toward the distal end side with respect to the operating portion 100.

### (Configuration of Operating Portion)

As shown in FIG. 3, the operating portion 100 has an operating portion main body (handle) 101, a slider (first operating portion) 102, and a distal end portion 104.

The operating portion main body 101 is formed in a rod shape extending in the direction of the longitudinal axis C1, and a finger hook portion 101a is provided at the proximal end portion. A slit 101b extending in the radial direction is formed in the operating portion main body 101. The slit 101b is formed to have a front end surface 101c and a proximal end surface 101d.

The slider 102 is provided so as to be inserted through the operating portion main body 101. The slider 102 can slide (forward and backward) in the longitudinal axis C1 direction with respect to the operating portion main body 101. In the present embodiment, when the slider 102 moves forward or backward in the longitudinal axis C1 direction, the operation wire 62 and the hook 62a fixed to the distal end of the operation wire 62 move forward or backward together with the slider 102.

Further, in a state where the operation wire 62 and the connecting rod 3 are connected, the arm member 11 of the clip 10 can move forward or backward together with the operation wire 62 by the forward or backward operation of the operation wire 62. As a result, the pair of first arm 12 and second arm 13 of the arm member 11 can be opened or closed.

The slider 102 is formed in a cylindrical shape. A concave portion 102a is formed on the outer peripheral surface of the slider 102 over the entire circumference. The slider 102 is formed with a flange portion 102b, a concave portion 102a, and a flange portion 102c in this order from the distal end side to the proximal end side in the axial direction C1. The pair of collar portions 102b and 102c have an elliptical shape when viewed in the radial direction of the slider 102. As a result, the slider 102 can be easily grasped, and space can be saved when packing the operating portion 100 of the endoscope clip 1.

By engaging the slider 102 with the slit 101b of the operating portion main body 101, the movement range of the slider 102 in the longitudinal axis C1 direction with respect to the operating portion main body 101 is limited. More specifically, the movable region of the slider 102 is limited between the distal end surface 101c and the proximal end surface 101d of the slit 101b.

The slider 102 is provided with a fixing portion (not shown) to which the proximal end of the operation wire 62 is fixed. In the present embodiment, the position where the fixing portion is provided on the slider 102 is not particularly limited. For example, the fixing portion may be provided at the distal end of the flange portion 102b of the slider 102.

The distal end portion 104 is formed on the distal end side of the operating portion main body 101 and has a pair of legs 104A and 104B. The pair of legs 104A and 104B are formed so as to be separated from each other at a predetermined distance in the radial direction of the operating portion 100. Further, each of the pair of legs 104A and 104B is formed with a convex portion 103 protruding outward in the radial direction of the operating portion 100 from the outer peripheral surface. FIG. 3 shows a state in which the pair of legs 104A and 104B are inserted through the opening on the proximal end side of the connecting member 80, and the convex portion 103 provided on the pair of legs 104A and 104B is engaged with the proximal concave portion 84 of the connecting member 80. In the present embodiment, the pair of convex portions 103 are formed having a trapezoidal cross-sectional shape, for example, as shown in FIG. 3. By forming the pair of convex portions 103 as described above, when the operator moves the connecting member 80 relative to the operating portion 100 (forward or backward), the pair of convex portions 103 can easily pass over the distal end concave portion 83 or the proximal end concave portion 84.

In the present embodiment, in consideration of the point that the operation wire 62 is inserted, the region from the distal end surface 104C of the distal end portion 104 of the operating portion 100 to the distal end surface 101c of the slit 101b of the operating portion main body 101 is defined as the sheath (second sheath) 66B through which the operation wire 62 is inserted. In other words, in the present embodiment, the operation wire 62 is connected to the operating portion 100 by inserting the sheath 66A, the first lumen 81 of the connecting member 80, a part of the second lumen 82 of the connecting member 80, and the sheath 66B from the distal end side of the medical device 1. In the present embodiment, the above-described region through which the operation wire 62 is inserted is defined as the sheath 66. More specifically, in the present embodiment, the sheath 66 is configured by connecting the sheath 66A and the sheath 66B by the connecting member 80, and is defined as a region from a position where the fixing portion is arranged when the slider 102 is moved forward to the maximum (for example, the distal end surface 101c of the slit 101b of the operating portion main body 101) to the distal end surface of the sheath 66A.

Based on the above definition, in the present embodiment, the distance from the proximal end surface of the sheath 66A to the distal end surface 104C of the operating portion 100, which is the distal end surface of the sheath 66B, along the longitudinal axis C1 direction is defined as the distance ΔL between the sheath 66A and the sheath 66B. The distance from the distal end surface of the sheath 66A to the distal end surface 101c of the slit 101b of the slider 102 is defined as the length L of the sheath 66.

Therefore, although the respective lengths of the sheath 66A and the sheath 66B do not change, the length L of the sheath 66 can be adjusted by adjusting the distance ΔL between the sheath 66A and the sheath 66B.

In the present embodiment, as shown in FIG. 3, the state in which the operating portion 100 and the connecting member 80 are engaged with each other by arranging the pair of convex portions 103 provided on the distal end portion 104 of the operating portion 100 in the proximal end concave portion 84 of the connecting member 80 is referred to as a first state of the sheath 66. In the first state of the sheath 66, the length L of the sheath 66 is the first length L1. Further, in the first state of the sheath 66, even if the slider 102 is moved toward the distal end side until it comes into contact with the distal end surface 101c of the slit 101b, as shown in FIG. 3, the hook 62a connected to the distal end of the operation wire 62 is located on the proximal end side of the distal end opening of the sheath 66A, that is, in a state of being accommodated inside the sheath 66A.

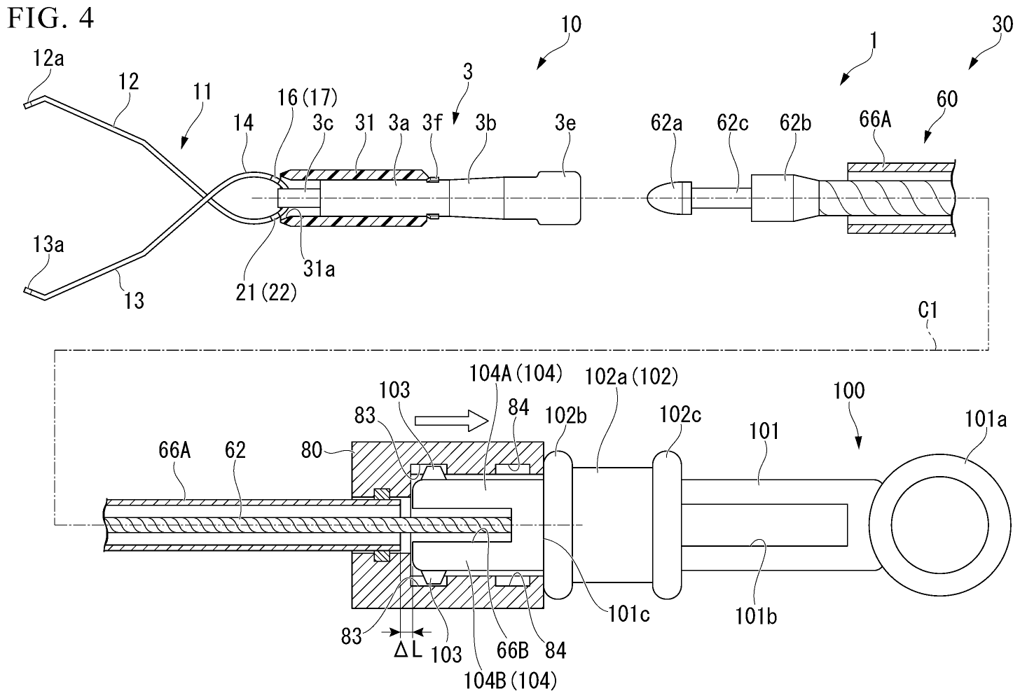

On the other hand, as shown in FIG. 4, by moving the connecting member 80 toward the proximal end side with respect to the operating portion 100, the pair of convex portions 103 can be arranged in the distal end concave portion 83 of the connecting member 80. At this time, the distance ΔL between the sheath 66A and the sheath 66B is reduced by the distance between the proximal end concave portion 84 and the distal concave portion 83. By moving the connecting member 80 to the proximal end side, the sheath 66A fixed to the connecting member 80 is moved to the proximal end side together with the connecting member 80. That is, the distal end opening of the sheath 66A is also moved to the proximal end side with respect to the hook 62a. As a result, as shown in FIG. 4, the hook 62a is in a state of protruding from the distal end opening of the sheath 66A.

In the present embodiment, a state in which a pair of convex portions 103 provided on the distal end portion 104 of the operating portion 100 are arranged in the distal end concave portion 83 of the connecting member 80 and the operating portion 100 and the connecting member 80 are engaged with each other is referred to as a second state of the sheath 66. Therefore, in the second state of the sheath 66, the length ΔL between the sheath 66A and the sheath 66B is reduced, so that the length of the sheath 66 changes from the length L1 in the first state to the second length L2, which is smaller than the length L1. Further, in the second state of the sheath 66, the hook 62a protrudes from the distal end opening of the sheath 66A, so that it becomes possible to engage with the notch 3d of the connecting rod 3 protruding from the opening on the proximal end side of the holding tube 31.

### (Operation to Load the Clip to the Applicator)

Hereinafter, the operation of loading the clip 10 according to the present embodiment to the applicator 30 will be described with reference to FIGS. 3 to 8.

FIG. 3 is a diagram showing an initial state of the applicator 30 according to the present embodiment. In FIG. 3, the pair of convex portions 103 of the operating portion 100 are arranged in the proximal end concave portion 84 of the connecting member 80, so that the operating portion 100 and the connecting member 80 are engaged with each other. In this state, the sheath 66 is in the first state in which the length is the first length L1. On the other hand, even if the slider 102 of the operating portion 100 comes into contact with the distal end surface 101c of the slit 101b, that is, is moved to the position on the most distal end side in the movable region, the hook 62a provided on the distal end side of the operation wire 62 is in the accommodation position accommodated in the sheath 66A.

From the state shown in FIG. 3, when the operator grasps the connecting member 80 and moves to the proximal end side with respect to the operating portion 100, the distal end surface of the proximal end concave portion 84 and the pair of convex portions 103 come into contact with each other. When the operator moves the connecting member 80 toward the proximal end while the distal end surface of the proximal end concave portion 84 and the pair of convex portions 103 are in contact with each other, a force is generated in which the distal end surface of the proximal end concave portion 84 presses against the pair of convex portions 103. This pressing force acts on the pair of legs 104A and 104B of the distal end portion 104 of the operating portion 100 to bring the pair of legs 104A and 104B close to each other. As a result, the width of the distal end portion 104 of the operating portion 100 in the radial direction is reduced, and the distal end surface of the proximal end concave portion 84 can be overcome. That is, by the operation of moving the connecting member 80 to the proximal end side of the connecting member 80 by the operator, as shown in FIG. 4, the connecting member 80 can be moved toward the proximal end side with respect to the operating portion 100 until the pair of convex portions 103 enter the distal end concave portion 83 of the connecting member 80.

As shown in FIG. 4, a pair of convex portions 103 of the operating portion 100 are arranged in the distal end concave portion 83 of the connecting member 80, and the operating portion 100 and the connecting member 80 are engaged with each other. In this state, as described above, the sheath 66 is in the second state in which the length is the second length L2. On the other hand, the hook 62a provided on the distal end side of the operation wire 62 is in a protruding position protruding from the opening on the distal end side of the sheath 66A, while the slider 102 of the operating portion 100 is in the position closest to the distal end side. Therefore, in the state shown in FIG. 4, the operator can engage the hook 62a with the notch 3d of the connecting rod 3. This completes the preparatory operation for loading the clip 10 according to the present embodiment to the applicator 30.

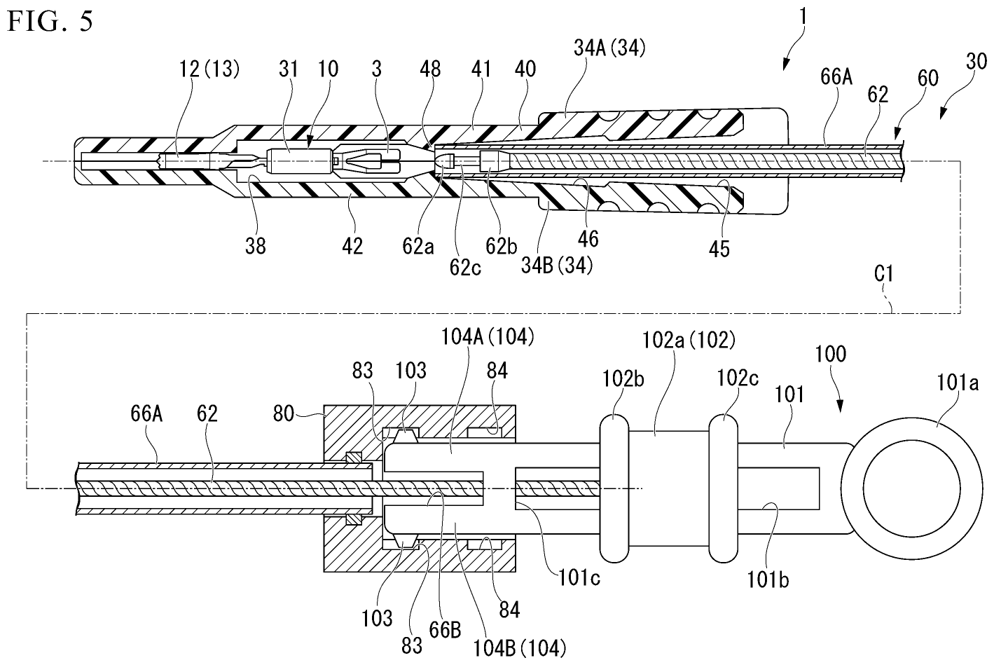

Next, the operator operates the applicator 30 and executes an operation of loading the clip 10 accommodated in the clip cartridge 40 to the applicator 30. As shown in FIG. 5, the operator inserts the applicator 30 into the clip cartridge 40 until the sheath 66A abuts on the stepped portion 48 of the clip cartridge 40. In this process, in order to prevent the hook 62a from unintentionally contacting the inside of the clip cartridge 40, as shown in FIG. 5, the operator can temporarily return the hook 62a to the accommodation position by slightly pulling the slider 102 back toward the proximal end side.

The operator grasps and compresses the pair of squeezing portions 34A and 34B of the compressing portion 34 of the clip cartridge 40. By this operation, as shown in FIG. 6A, the pair of compressed portions 34A and 34B of the compression portion 34 are elastically deformed so as to approach each other, and the position of the sheath 66A in the longitudinal axis C1 direction is fixed. Then, the operator pushes the slider 102 toward the distal end side from the state shown in FIG. 5 until the slider 102 comes into contact with the distal end surface 101c of the slit 101b. As a result, the hook 62a moves from the accommodation position to the protruding position, passes over the step portion 48 of the clip cartridge 40, and is inserted into the inside of the connecting rod 3 through the through hole of the deformed portion 3e on the proximal end side of the connecting rod 3.

In the present embodiment, when the operator moves the slider 102 to the position closest to the distal end side, the hook 62a is engaged with the notch 3d of the connecting rod.

However, the present invention is not limited to this. For example, in the process in which the slider 102 is advanced along the slit 101b to a position where it abuts on the distal end surface 101c of the slit 101b, it is also possible for the hook 62a to enter and engage with the notch 3d of the connecting rod 3 before the slider 102 abuts on the distal end surface 101c. At this time, when the hook 62a enters the cutout portion 3d of the connecting rod 3 and is engaged, the position of the slider 102 in the operating portion 100 in the slit 101b can be regarded as the most advanced position of the slider 102.

In the present embodiment, as shown in FIG. 6B, when the connecting portion of the connecting rod 3 and the hook 62a is in a position protruding from the sheath 66A, the hook 62a may unintentionally fall off from the side of the notch 3d of the connecting rod 3 by the connecting rod 3 rotating in the direction intersecting the longitudinal axis C1 direction along the direction indicated by the arrow D. In order to prevent such a situation, in a state where the slider 102 is in contact with the distal end surface 101c of the slit 101b and the hook 62a is engaged with the notch 3d of the connecting rod 3 (see FIG. 6A), the operator needs to accommodate the connecting portion between the hook 62a and the connecting rod 3 in the sheath 66A before completely removing the applicator 30 from the clip cartridge 40.

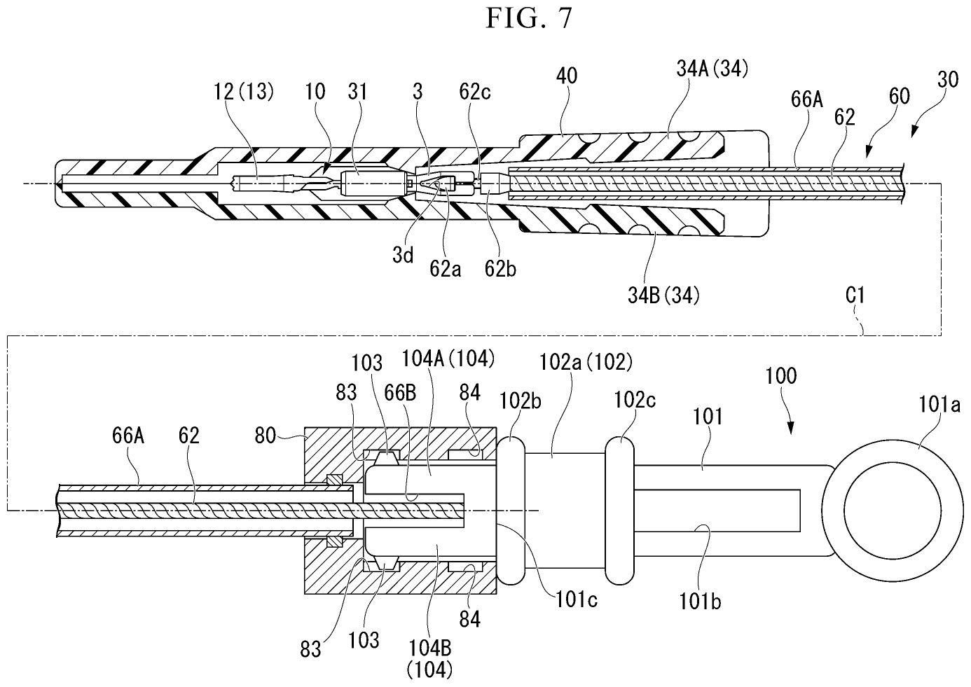

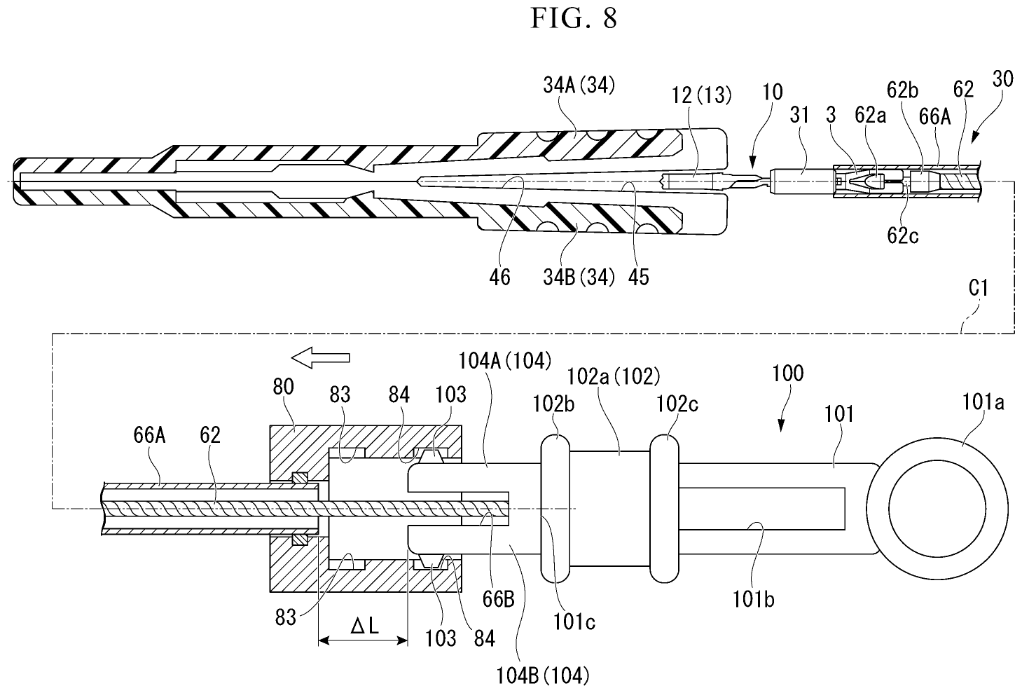

In order to accommodate the connecting portion between the hook 62a and the connecting rod 3 in the sheath 66A, for example, the following operations can be performed. First, as shown in FIG. 7, the operator releases the compressed state of the pair of compressed portions 34A and 34B of the compressed portion 34 of the clip cartridge 40, does not operate the slider 102, and grasps the operating portion main body 101 of the operating portion 100 and slightly pulls the entire operating portion 100 back to the proximal end side. By this operation, the connecting portion between the hook 62a and the connecting rod 3 is pulled back to a position closer to the proximal end side than the step portion 48 of the clip cartridge 40. After that, the operator moves the connecting member 80 toward the distal end side with respect to the operating portion 100, so that the pair of convex portions 103 of the distal end portion 104 of the operating portion 100 can be arranged in the proximal end concave portion 84 of the connecting member 80. In other words, the operator moves the connecting member 80 toward the distal end side with respect to the operating portion 100, so that the sheath 66 can be transitioned from the second state to the first state, and the length L of the sheath 66 can be adjusted from the second length L2 to the first length L1. As a result, as shown in FIG. 8, the connecting portion between the hook 62a and the connecting rod 3 can be arranged inside the sheath 66A even when the slider 102 is in contact with the distal end surface 101c of the slit 101b.

In this state, as shown in FIG. 9, the clip 10 is in contact with the sheath 66A. More specifically, the proximal end surface of the holding tube 31 of the clip 10 is in contact with the distal end surface of the sheath 66A. In this embodiment, the holding tube 31 of the clip 10 and the sheath 66A may not be in close contact with each other due to manufacturing tolerances of parts, errors during operation, or the like, and the distance between them may be almost zero. In this case, since the clip 10 and the applicator 30 are connected by the engagement between the hook 62a and the connecting rod 3, the clip 10 does not unintentionally fall off from the applicator 30. In the present specification, the case where the distance between the clip 10 and the sheath 66A is almost zero is also included in “the clip 10 comes into contact with the sheath 66A”.

Further, a cup-shaped cover member can be arranged at the distal end of the sheath 66A to support the holding tube 31. At this time, the clip 10 may not come into direct contact with the sheath 66A, but may come into contact with any part of the cover member. In this case, since the proximal end portion of the clip 10, more specifically, a part of the proximal end side of the holding tube 31, is received by the cup-shaped cover member, the clip 10 is more stably loaded to the applicator 30. In the present specification, the case where the clip 10 is supported by the cover member provided at the distal end portion of the sheath 66A is also included in “the clip 10 comes into contact with the sheath 66A”.

After that, the operator can grasp the operating portion 100 and integrally remove the applicator 30 and the clip 10 connected to the applicator 30 from the clip cartridge 40.

As a result, the operation of loading the clip 10 according to the present embodiment to the applicator 30 and configuring the medical device 1 is completed.

### (Procedure by Medical Device 1)

Hereinafter, a procedure for ligating the target tissue T using the medical device 1 according to the present embodiment will be described with reference to FIGS. 9 to 12B.

As shown in FIG. 9, when the clip 10 is taken out from the clip cartridge 40 with the clip 10 loaded to the applicator 30, the first arm 12 and the second arm 13 of the arm member 11 of the clip 10 are in an open state separated from each other by their own elastic restoring force. At this time, in the operating portion 100 on the proximal end side, the slider 102 is in the most advanced position where it abuts on the distal end surface 101c of the slit 101b. Further, since the pair of convex portions 103 of the distal end portion 104 of the operating portion 100 are arranged in the proximal end concave portion 84 of the connecting member 80, the sheath 66 is in the first state having the first length L1. That is, the connecting portion between the hook 62a and the connecting rod 3 does not protrude from the opening on the distal end side of the sheath 66A.

In the procedure according to the present embodiment, the operator does not move the connecting member 80 again and shift the sheath 66 from the first state to the second state until the operation of ligating one target tissue T is completed.

As shown in FIG. 9, the elastic restoring force of the first arm 12 and the second arm 13 of the arm member 11 of the clip 10 has an effect of moving the holding tube 31 toward the proximal end side. Therefore, the holding tube 31 is in contact with the distal end of the sheath 66A. However, since the holding tube 31 has an outer diameter larger than the inner diameter of the sheath 66A, it does not enter the sheath 66A.

The operator inserts an endoscope (not shown) into the patient's body. Then, the operator can insert the medical device 1 from the proximal end of the endoscope channel and protrude the medical device 1 from the distal end of the endoscope channel.

In this process, in order to smoothly insert the endoscope channel through the medical device 1, it is necessary to temporarily close the first arm 12 and the second arm 13 of the arm member 11 in the open state. In this case, although not shown, for example, the operator pulls back the slider 102 of the operating portion 100 on the proximal end side, and pulls the portion located on the distal end side of the intersection of the first arm 12 and the second arm 13 into the holding tube 31. Thereby, the first arm 12 and the second arm 13 can be closed. At that time, the pair of protrusions 16 and 17 provided in the intermediate portion 14 of the arm member 11 are engaged with the inner wall of the holding tube 31, and the elastic restoring force of the first arm 12 and the second arm 13 can restrict the movement of the arm member 11 toward the distal end side with respect to the holding tube 31.

When the operator guides the medical device 1 through the channel of the endoscope to the vicinity of the target tissue T and pushes the slider 102 toward the distal end side, the first arm 12 and the second arm 13 of the arm member 11 can be changed to the open state again.

In the present embodiment, for convenience of explanation, an example in which the slider 102 is in the most advanced position where the slider 102 abuts on the distal end surface 101c of the slit 101b will be described as shown in FIG. 9, but the present invention is not limited to such a configuration. For example, when adjusting the opening widths of the first arm 12 and the second arm 13 of the arm member 11 to the optimum values in order to correspond to the target tissue T, by moving the slider 102 forward and backward, the opening width of the first arm 12 and the second arm 13 can be increased or decreased. Even in such a case, since the sheath 66 maintains the first state in which the length L is the first length L1, even if the slider 102 is moved forward and backward, the engaging portion between the hook 62a and the connecting rod 3 does not protrude from the distal end opening of the sheath 66A.

As shown in FIG. 9, the operator can operate an endoscope (not shown) to adjust the direction and orientation of the arm member 11 of the clip 10 and press the arm member 11 toward the target tissue T. By such an operation, the target tissue T can be positioned between the first arm 12 and the second arm 13 of the arm member 11 in the open state. When the operator confirms that the target tissue T is located between the first arm 12 and the second arm 13, the operator can operate the endoscope to hold the target tissue T by the first arm 12 and the second arm 13 of the arm member 11.

After confirming that the target tissue T is located between the first arm 12 and the second arm 13, the operator grasps the operating portion main body 101 and pulls the slider 102 back to the proximal end side. At this time, the first arm 12 and the second arm 13 move to the proximal end side together with the operation wire 62. In such a state, while the first arm 12 and the second arm 13 are in contact with the tapered surface 31a provided on the distal end side of the holding tube 31, the first arm 12 is elastically deformed toward the second arm 13, and the second arm 13 is elastically deformed toward the first arm 12. As a result, as shown in FIG. 10, the distal end of the first arm 12 and the distal end of the second arm 13 come close to each other, and the opening width of the arm member 11 is reduced.

In this state, the operator presses the holding tube 31 with the first arm 12 and the second arm 13 in contact with the distal end of the holding tube 31 by the force of pulling the slider 102 back to the proximal end side. The pressing force provided by the first arm 12 and the second arm 13 that acts on the holding tube also acts on the sheath 66A that is in contact with the holding tube 31. Therefore, the above-described pressing force also acts on the connecting member 80 fixed to the sheath 66A, and has an effect of moving the connecting member 80 toward the proximal end side.

That is, in the present embodiment, by the above-described pressing force acting on the connecting member 80, the connecting member 80 has an effect of pressing the pair of convex portions 103 and elastically deforming the pair of legs 104A and 104B of the distal end portion 104 of the operating portion 100.

However, in the present embodiment, the force required to elastically deform the pair of legs 104A and 104B of the distal end portion 104 of the operating portion 100 is set to be larger than the above-described pressing force. Therefore, the above-described pressing force elastically deforms the pair of legs 104A and 104B, and the connecting member 80 cannot be moved to the proximal end side with respect to the operating portion 100.

In other words, in the present embodiment, the pair of legs 104A and 104B (including the pair of convex portions 103) of the distal end portion 104 of the operating portion 100 can generate a regulatory force that restricts the connecting member 80 from moving toward the proximal end side with respect to the operating portion 100. This restricting force is generated by the elastic restoring force of the pair of legs 104A and 104B. That is, in the present embodiment, the pair of legs 104A and 104B (including the pair of convex portions 103) of the distal end portion 104 of the operating portion 100 are a restricting portion that regulates the movement of the connecting member 80 toward the proximal end side with respect to the operating portion 100.

In the present embodiment, this restricting force is set to be larger than the maximum value of the traction force for pulling the slider 102 back to the proximal end side by the operator, in order to grasp the target tissue T, in the process of transitioning the first arm 12 and the second arm 13 of the arm member 11 from the open state to the closed state. Therefore, in the process in which the operator pulls back the slider 102 to the proximal end side and grasps the target tissue T, the connecting member 80 does not move to the proximal end side with respect to the operating portion 100. In other words, in this process, the sheath 66A does not move at the proximal end side with respect to the sheath 66B.

That is, the arm member 11 changes from the open state to the closed state in a state where the target tissue T is located between the first arm 12 and the second arm 13. As a result, as shown in FIG. 10, the target tissue T is held by the first arm 12 and the second arm 13. In the present embodiment, as shown in FIG. 10, until the target tissue T is grasped by the first arm 12 and the second arm 13 and a pair of protrusions 16 and 17 formed in the intermediate portion 14 of the arm member 11 are located in the holding tube 31 on the distal end side of the proximal end, that is, until the first arm 12 and the second arm 13 of the arm member 11 are completely closed, the operator can move the arm member 11 toward the distal end side with respect to the holding tube 31 by pushing the slider 102 toward the distal end side again. As a result, the opening width between the first arm 12 and the second arm 13 of the arm member 11 can be increased. In other words, the arm member 11 can be transitioned to the open state shown in FIG. 9. In the embodiment shown in FIG. 9, the sheath 66A is in contact with the holding tube 31, but the sheath 66A may not be in contact with the holding tube 31. In either case, since the connecting portion between the hook 62a and the connecting rod 3 is arranged inside the spring 86, the connection between the hook 62a and the connecting rod 3 is maintained.

Therefore, in the present embodiment, in the process of pulling the slider 102 back to the above-described state, by operating the slider 102 until the first arm 12 and the second arm 13 of the arm member 11 are completely closed (a state in which the pair of protrusions 16 and 17 are located on the proximal end side of the opening on the proximal end side of the holding tube 31), the operator can adjust the open/closed state and orientation of the clip 10 again and re-grasp the target tissue T.

If the operator can confirm that the target tissue T is grasped by the arm member 11 of the clip 10 in a desired state, the operator proceeds to the operation of locking the grasped state of the clip 10 of the endoscope clip 1.

At this time, by pulling the slider 102 further toward the proximal end side, the operator pulls back the pair of protrusions 16 and 17 provided in the intermediate portion 14 of the arm member 11 to a position protruding from the opening on the proximal end side of the holding tube 31. When the pair of protrusions 16 and 17 are located closer to the proximal end side than the opening on the proximal end side of the holding tube 31, the pair of protrusions 16 and 17 can abut and engage with the proximal end surface of the holding tube 31. As a result, the arm member 11 is restricted from moving toward the distal end side with respect to the holding tube 31. That is, even if the force for pulling the slider 102 back to the proximal end side by the operator is released, the pair of protrusions 16 and 17 are locked to the distal end side with respect to the proximal end surface of the holding tube 31.

Therefore, the state in which the target tissue T is bound and grasped by the clip 10 is locked. In the present embodiment, the closed state of the arm member 11 includes a state in which the target tissue T is tied up at the root by the first arm 12 and the second arm 13, and the distance between the first arm 12 and the second arm 13 is approximately 0.

After this, the operator separates the clip 10 to which the target tissue T is ligated from the applicator 30. More specifically, as shown in FIG. 12A, in a locked state in which the pair of protrusions 16 and 17 are locked to the distal end side with respect to the proximal end surface of the holding tube 31, when the operator further pulls the slider 102 back to the proximal end side, the force for pulling back the slider 102 acts on the hook portion 3c of the connecting rod 3. As shown in FIG. 12B, when the force for pulling back the slider 102 exceeds the limit that the hook portion 3c can withstand, the connecting rod 3 is plastically deformed so that the hook portion 3c is curved as shown in FIG. 12B. As a result, the engagement between the hook portion 3c of the connecting rod 3 and the intermediate portion 14 of the arm member 11 is released.

Thereby, the arm member 11 to which the target tissue T is ligated is placed in the body together with the holding tube 31. On the other hand, the connecting rod 3 is removed from the body while being engaged with the hook 62a.

After the clip 10 ligated with the target tissue T is placed in the body, the operator can operate the endoscope and take out the medical device 1 from the channel of the endoscope. After that, the operator takes necessary measures and completes a series of procedures. For example, the operator can take the medical device 1 out of the body and then remove the connecting rod 3 from the distal end of the hook 62a and dispose of it according to the method shown in FIG. 6B described above. On the other hand, the applicator 30 including the hook 62a can be sterilized as necessary, and a new clip 10 can be loaded and reused by the loading method shown in the present embodiment.

### (Effect of Medical Device 1)

According to the medical device 1 of the present embodiment, in the applicator 30, the sheath 66A and the sheath 66B are connected by a connecting member 80. By the operator moving the connecting member 80 toward the proximal end side toward the sheath 66B, the sheath 66A fixed to the connecting member 80 moves toward the sheath 66B, and the distance between the sheath 66A and the sheath 66B decreases. Thereby, the sheath 66 of the applicator 30 is reduced in length from the first length L1 to the second length L2. As a result, the hook 62a provided at the distal end of the operation wire 62 can move relative to the sheath 66A from the accommodation position in the sheath 66A to the protruding position protruding from the distal end opening of the sheath 66A. As a result, the operator can operate the slider 102 of the operating portion 100 and engage the hook 62a with the notch 3d of the connecting rod 3 to connect the clip 10 and the applicator 30.

On the other hand, when the clip 10 and the applicator 30 are connected, the operator operates the connecting member 80 again and moves the connecting member 80 to the distal end side away from the sheath 66B, thereby separating the sheath 66A from the sheath 66B. By this operation, the connecting portion between the hook 62a and the connecting rod 3 moves from the protruding position protruding from the sheath 66A to the accommodation position in the sheath 66A. As a result, the operator moves the slider of the operating portion 100 to the most advanced position in the procedure of ligating the target tissue T by using the medical device 1 configured by loading the clip 10 to the applicator 30. However, it is possible to prevent the connecting portion between the hook 62a and the connecting rod 3 from protruding from the distal end opening of the sheath 66A and the clip 10 from being unintentionally dropped from the applicator 30. That is, according to the medical device 1 of the present embodiment, in the procedure for ligating the target tissue T, the state and orientation of the clip 10 can be adjusted and the target tissue T can be re-grasped by moving the slider 102 forward and backward.

Further, when the procedure for ligating the target tissue T is completed, the operator can load the new clip 10 by removing the connecting rod 3 connected to the hook 62a from the body and then discarding it. That is, according to the medical device 1 of the present embodiment, the clip 10 can be reloadable.

Therefore, according to the medical device 1 of the present embodiment, both the reloadability of the clip 10 and the re-grasping operation of the target tissue T can be achieved for the applicator 30.

According to the medical device 1 of the present embodiment, the connecting member 80 is configured by only one component and can be easily manufactured, so that the medical device 1 can be configured at low cost. Further, since the length of the sheath 66 can be adjusted only by moving the connecting member 80 forward and backward by the operation of the operator's hand, the influence of the meandering state of the endoscope in the body is small, and the operation of the operator becomes simple.

### (Modified Example of the First Embodiment)

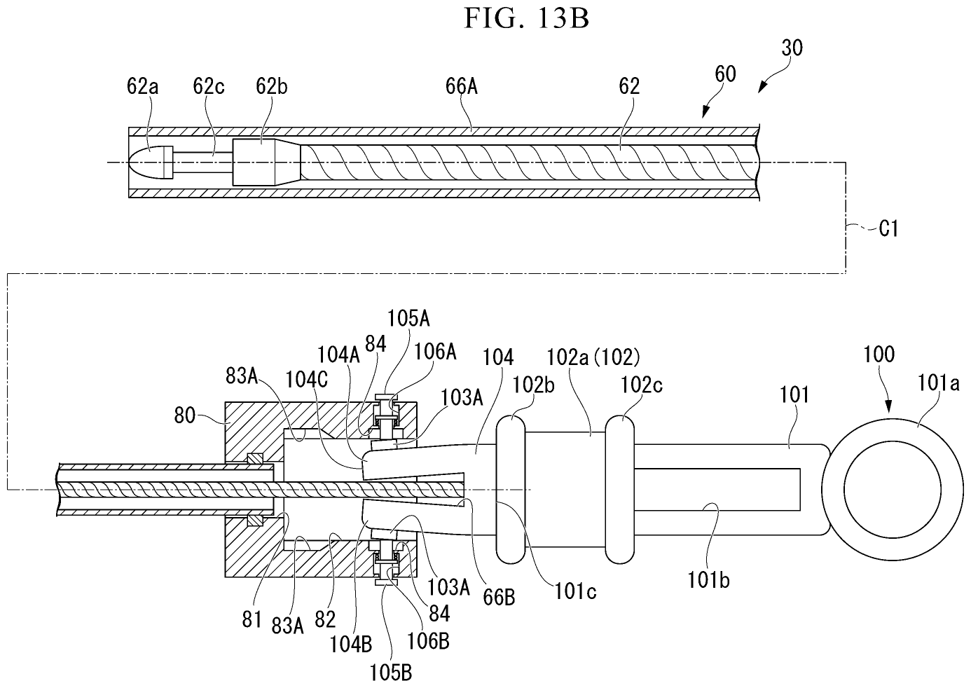

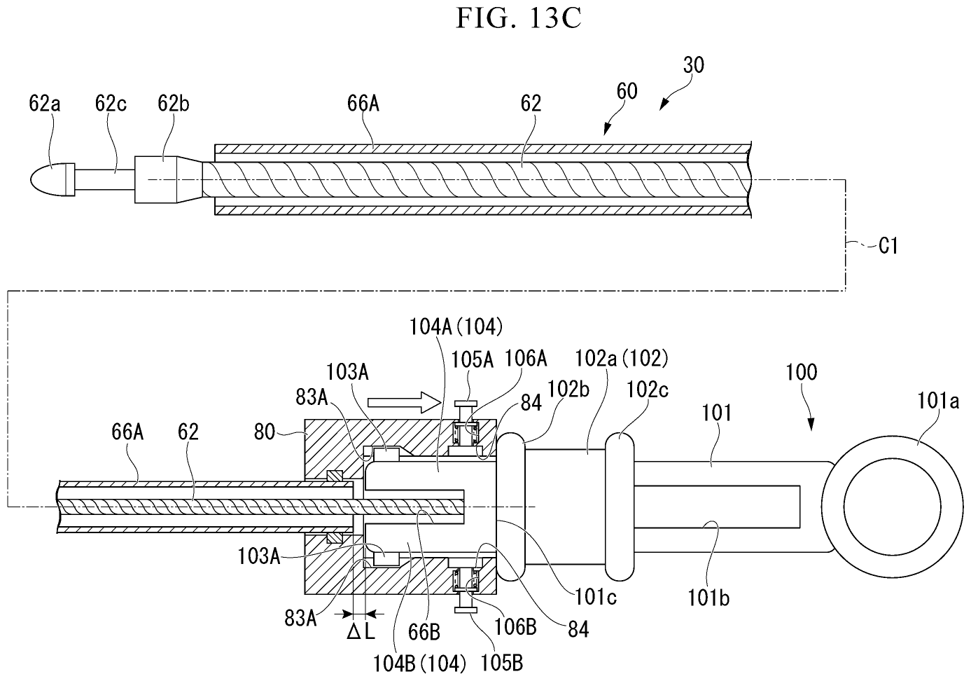

Hereinafter, the configuration of a modified example of the applicator 30 according to the present embodiment will be described with reference to FIGS. 13A to 13C. Hereinafter, the same configuration as the applicator 30 according to the first embodiment will be described with the same reference numerals, the description thereof will be omitted, and the points different from those of the above-described embodiment will be mainly described.

As shown in FIG. 13A, the applicator 30 according to the present modification has a different configuration in which the connecting member 80 and the distal end portion 104 of the operating portion 100 are engaged with each other as compared with the applicator 30 according to the first embodiment described above. More specifically, in the applicator 30 according to the present modification, the pair of convex portions 103A provided on the pair of legs 104A and 104B of the distal end portion 104 of the operating portion 100 are formed to have a rectangular cross-sectional shape.

The proximal end side of the distal end concave portion 83A in the connecting portion 80 according to this modification is chamfered. Therefore, in the distal end concave portion 83 according to the first embodiment described above, the proximal end surface is formed orthogonal to the longitudinal axis C1, whereas as shown in FIG. 13A, the distal end concave portion 83A in the connecting portion 80 according to this modification is formed with a proximal end surface inclined to the longitudinal axis C1. Further, as shown in FIG. 13A, a through hole communicating with the outer peripheral surface of the connecting portion 80 is formed in the proximal end concave portion 84 of the connecting portion 80 according to the present modification. The pair of pins 105A and 105B are inserted into a through hole formed in the proximal end concave portion 84 of the connecting portion 80, and are urged outward in the radial direction by the pair of elastic members 106A and 106B.

Since the applicator 30 according to the present modification has the above-described configuration, as shown in FIG. 13A, when the pair of convex portions 103A are arranged in the proximal end concave portions 84 of the connecting member 80, in the direction along the longitudinal axis C1, a pair of convex portions 103A having a rectangular cross-sectional shape and a proximal end concave portion 84 having a rectangular groove shape come into contact with each other, and even if the force of the operator pushing the slider 102 toward the distal end or the force of pulling it back toward the proximal end side acts on the connecting member 80, the pair of convex portions 103A are regulated to move forward or backward over the proximal end concave portion 84. That is, even if the force for the operator to operate the slider 102 acts on the connecting member 80, the hook 62a provided at the distal end of the operation wire 62 is restricted from unintentionally protruding from the distal end opening of the sheath 66A.

In the applicator 30 according to this modification, the operator can push the pair of pins 105A inserted into the pair of through holes formed in the proximal end concave portion 84 inward in the radial direction of the connecting member 80. By this operation, as shown in FIG. 13B, the pair of pins 105A press the convex portions 103A formed on the pair of legs 104A and 104B against the elastic force of the pair of elastic members 106A and 106B. As a result, the pair of legs 104A and 104B of the distal end portion 104 bend (elastically deform) inward in the radial direction of the connecting member 80.

When the pair of legs 104A and 104B at the distal end portion 104 of the operating portion 100 according to this modification bend inward in the radial direction of the connecting member 80, the pair of convex portions 103A move inward in the radial direction of the connecting member 80 together with the pair of legs 104A and 104B. As a result, as shown in FIG. 13B, the pair of convex portions 103A are disengaged from the proximal end concave portion 84 of the connecting member 80, and the engagement between the pair of concave portions 103A and the proximal end concave portion 84 of the connecting member 80 is released.

In this state, as in the first embodiment described above, when the connecting member 80 is grasped and moved toward the proximal end side with respect to the operating portion 100, the pair of convex portions 103A can move toward the distal end side with respect to the connecting member 80. That is, according to the applicator 30 of the present modification, in a state where the operator pushes in the pair of pins 105A and 105B provided on the connecting member 80, by moving the connecting member 80 toward the proximal end, a pair of convex portions 103A provided on the distal end portion 104 of the operating portion 100 overcome the proximal end concave portion 84 of the connecting member 80, move toward the distal end side with respect to the connecting member 80, and enter the distal end concave portion 83A. As a result, as shown in FIG. 13C, the pair of convex portions 103A are engaged with the distal end concave portion 83A of the connecting member 80. In this state, the hook 62a connected to the distal end side of the operation wire 62 protrudes from the opening on the distal end side of the sheath 66A. Further, the pair of pins 105A and 105B inserted into the through holes of the proximal end concave portion 84 of the connecting member 80 are returned to the positions urged outward in the radial direction by the elastic force of the elastic members 106A and 106B.

After that, the operator can load the clip 10 to the applicator 30 and treat the target tissue T in the same manner as the applicator 30 according to the first embodiment described above.

Further, although not shown, according to the applicator 30 of the present modification, in a state where the pair of convex portions 103A are in contact with the distal end concave portion 83A, the operator can grasp the connecting member 80 and move the connecting member 80 to the distal end side with respect to the operating portion 100. When the operator moves the connecting member 80 toward the distal end side with respect to the operating portion 100, while the pair of convex portions 103A abut on the proximal end surface of the distal end concave portion 83A inclined with respect to the longitudinal axis C1, it can be moved to the proximal end side with respect to the operating portion 100. As a result, the pair of convex portions 103A can pass over the distal end concave portion 83A of the connecting member 80, move toward the proximal end side with respect to the connecting member 80, and engage with the proximal concave portion 84.

According to the applicator 30 of the present modification, similarly to the applicator 30 of the first embodiment described above, the operator can adjust the length L of the sheath 66 by adjusting the distance ΔL between the sheath 66A and the sheath 66B. As a result, the sheath 66 can be transitioned to the first state and the second state in the same manner as the applicator 30 according to the first embodiment described above.

According to the applicator 30 of the present modification, when the pair of convex portions 103A are engaged with the proximal concave portion 84 at the distal end portion 104 of the operating portion 100, even if the force of the operator moving the slider 102 forward and backward acts on the connecting member 80, it is possible to regulate that the pair of convex portions 103A and the proximal end concave portion 84 are disengaged from each other. Further, when the pair of convex portions 103A are engaged with the distal end concave portion 83A of the operating portion 100, the engagement between the pair of convex portions 103A and the distal end concave portion 83A can be easily disengaged by the force of the operator to move the connecting member 80 toward the distal end side with respect to the operating portion 100.

That is, according to the applicator 30 of the present modification, the state in which the hook 62a provided on the distal end side of the operation wire 62 is accommodated in the sheath 66A can be more reliably maintained as compared with the applicator 30 according to the first embodiment described above. In other words, according to the applicator 30 of the present modification, the operator can more reliably restrict the hook 62a from unintentionally protruding from the distal end opening of the sheath 66A by operating the slider 102.

### Second Embodiment

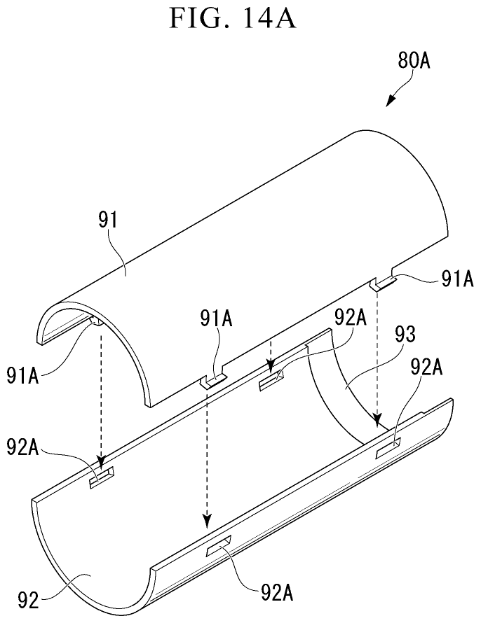

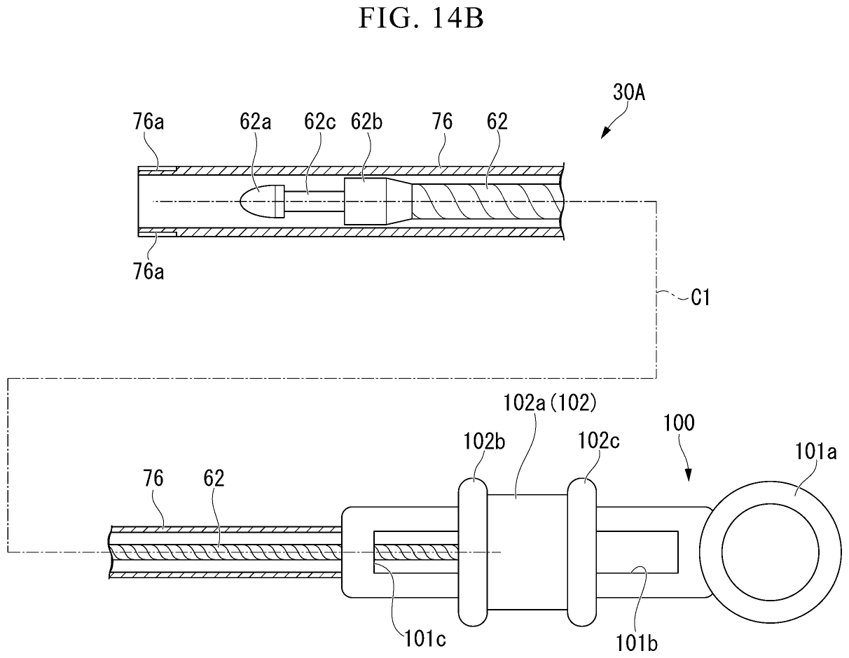

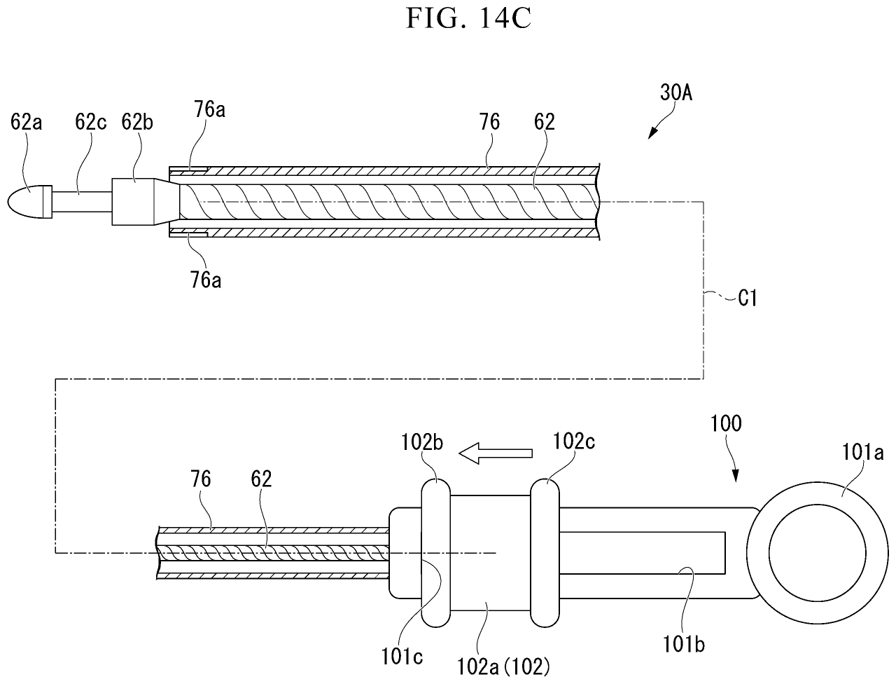

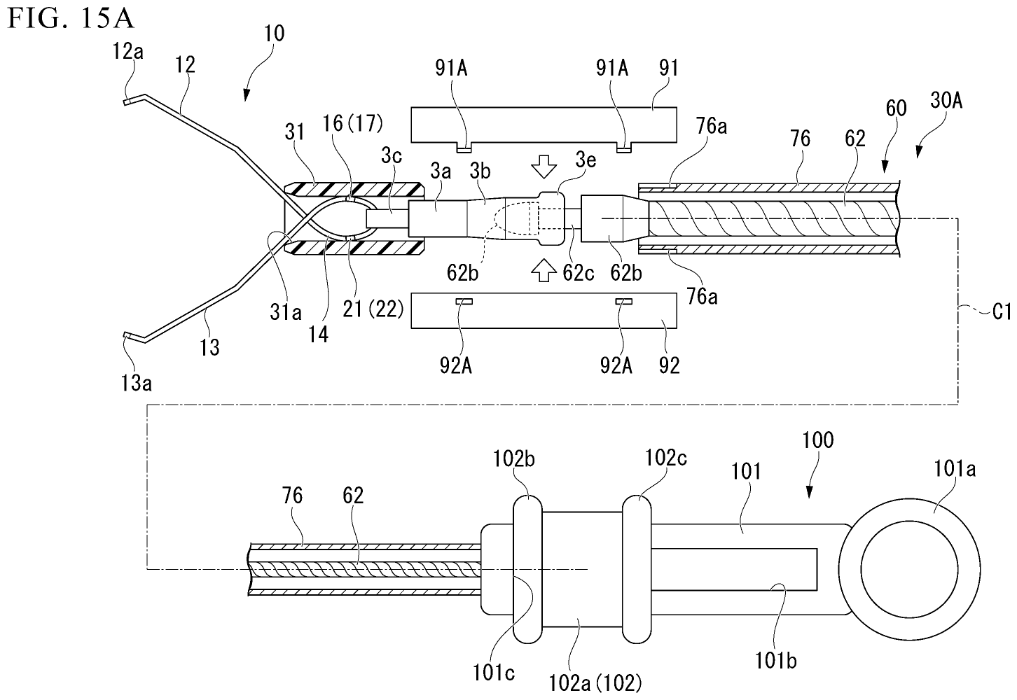

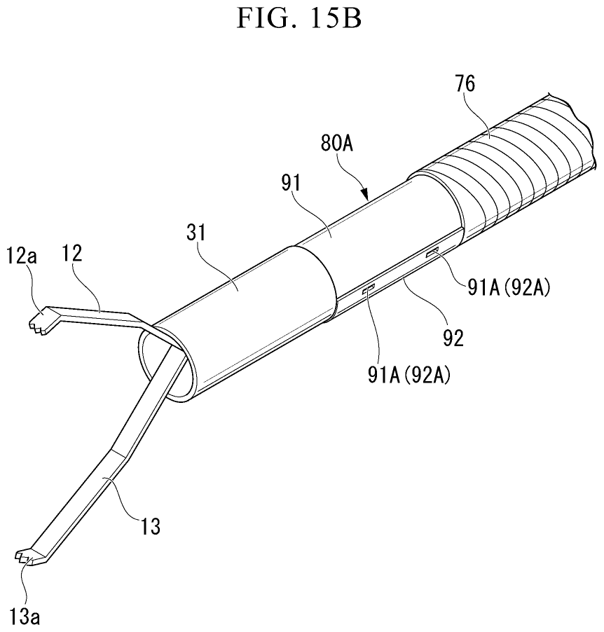

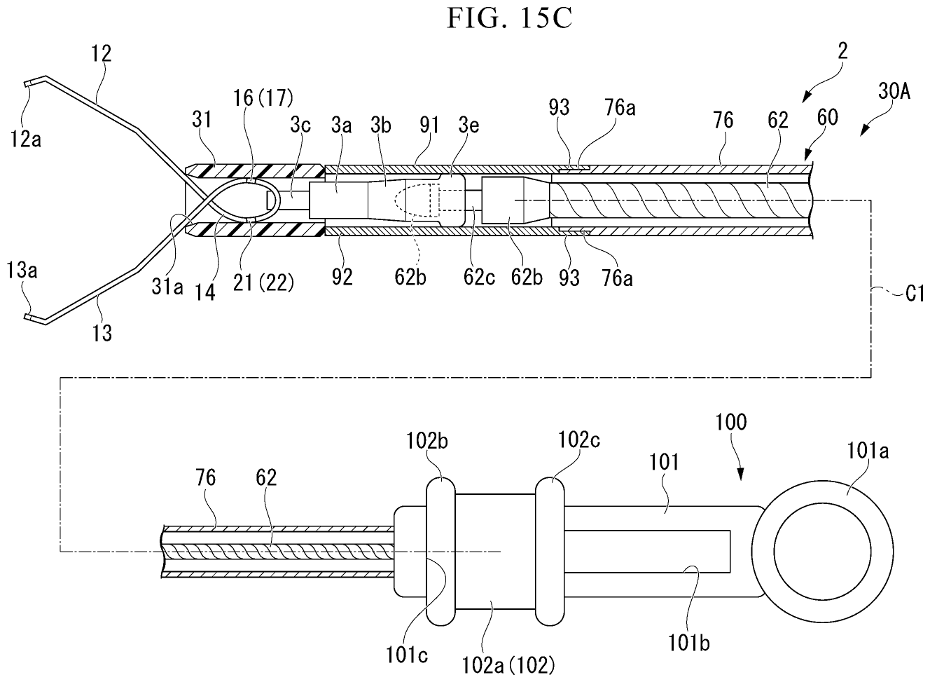

Hereinafter, the medical device according to the second embodiment of the present invention will be described with reference to FIGS. 14A to 15C. Hereinafter, the same configuration as the medical device 1 according to the first embodiment will be described with the same reference numerals, the description thereof will be omitted, and the points different from those of the above-described embodiment will be mainly described.

FIG. 14A is a perspective view showing the configuration of the protective member 80A of the medical device 2 according to the present embodiment. FIGS. 14B and 14C are side view partial cross-sectional views showing the configuration of the applicator 30A of the medical device 2 according to the present embodiment. FIGS. 15A to 15C are views showing an operation of loading the clip 10 according to the present embodiment to the applicator 30A.

Compared to the medical device 2 according to the first embodiment described above, the difference is that the operator attaches a protective member 80A externally instead of the connecting member 80 as shown in FIG. 15A to protect the connecting portion between the clip 10 and the applicator 30A. The medical device 2 according to the present embodiment has the clip 10 according to the first embodiment described above.

As shown in FIG. 14A, the protective member 80A according to the present embodiment is configured by fitting two members. Specifically, the protective member 80A is a tube-shaped member formed by fitting the upper member 91 and the lower member 92. As shown in FIG. 14A, the upper member 91 and the lower member 92 have a semicircular cross-sectional shape when viewed from the longitudinal axis direction. In the upper member 91, four engaging claws 91A are formed at positions line-symmetrical with respect to the longitudinal axis. The lower member 92 has four engaging holes 92A formed at positions corresponding to the four engaging claws 91. However, the present invention is not limited to this. The upper member 91 and the lower member 92 may be engaged with each other, and the number and arrangement positions of the engaging claws 91A and the engaging holes 92A are not particularly limited.

In the present embodiment, the protective member 80A formed by engaging the upper member 91 and the lower member 92 is formed in a tube shape. The protective member 80A may have an inner diameter that does not allow disengagement with each other when the connecting portion between the hook 62a and the connecting rod 3 is arranged in the protective member 80A, and the numerical value is not specifically limited. Further, the protective member 80A may have an outer diameter sufficient to come into contact with the clip 10.

In the present embodiment, the upper member 91 and the lower member 92 have the same length in the longitudinal axis direction. Further, the lengths of the upper member 91 and the lower member 92 in the longitudinal axis direction need only be able to completely cover the connecting portion between the operation wire 62 and the clip 10, which will be described later, and for example, they may be larger than the sum of the length of the connecting rod 3 of the clip 10 and the lengths of the hook 62a, the fixing portion 62b, and the shaft portion 62c.

As shown in FIGS. 14A and 15C, in the protective member 80A according to the present embodiment, in order to engage with the sheath 76 of the applicator 30A, which will be described later, as shown in FIGS. 14A and 15C, a thin-walled portion 93 formed by cutting out a part is formed on the proximal end side. The material forming the protective member 80A is not particularly limited. For example, considering that it can be used repeatedly, the protective member 80A may be formed of a metal material having biocompatibility or a resin material having a certain strength.

FIGS. 14B and 14C are diagrams showing the configuration of the applicator 30A according to the present embodiment. The applicator 30A according to the present embodiment has an operation wire 62 and an operating portion 100, similarly to the applicator 30 according to the first embodiment described above. On the distal end side of the operation wire 62, the hook 62a having an arrowhead shape, the large diameter portion 62b, and the shaft portion 62c are integrally formed. In order to engage and support the applicator 30A with the protective member 80A, a support portion 76a is formed by cutting off a part of the distal end portion of the sheath 76. That is, in the present embodiment, the protective member 80A can be connected to the sheath 76 by bringing the thin portion 81 on the proximal end side of the protective member 80A into contact with the support portion 76a of the sheath 76 (see FIG. 15C). In the present embodiment, the region from the distal end of the applicator 30A (the distal end of the support portion 76a) to the distal end surface 101c of the slit 101b of the slider 102 is defined as the sheath 76.

In the present embodiment, as shown in FIGS. 14B and 14C, when moving the slider 102 to the position closest to the distal end side until the slider 102 of the operating portion 100 comes into contact with the distal end surface 101c of the slit 101b of the operating portion main body 101, the hook 62a can be moved from the accommodation position accommodated inside the sheath 76 to the protruding position protruding from the distal end opening of the sheath 76. That is, in the present embodiment, when the slider 102 is moved to the position closest to the distal end side, the clip 10 can be connected to the applicator 30A by engaging the hook 62a with the connecting rod 3 of the clip 10.

On the other hand, when the target tissue T is treated using the medical device 2 configured by connecting the clip 10 to the applicator 30A, depending on the position of the slider 102, the connecting portion between the clip 10 and the applicator 30A may be moved to a protruding position protruding from the distal end opening of the sheath 76. Therefore, in the present embodiment, it is necessary to prevent the clip 10 from being unintentionally dropped from the applicator 30A.

FIG. 15A is a diagram showing a state in which the clip 10 according to the present embodiment is loaded to the applicator 30A. As shown in FIG. 15A, the hook 62a of the operation wire 62 is engaged with each other in a state of being inserted into the notch 3d of the connecting rod 3. Further, the connecting portion between the operation wire 62 and the clip 10 is located at a protruding position protruding from the distal end opening of the sheath 76.

At this time, the operator arranges the upper member 91 and the lower member 92 on the support portion 76a formed at the distal end of the sheath 76 in a state of facing each other with respect to the connecting portion between the operation wire 62 and the clip 10 arranged at the protruding position, and each of the plurality of engaging claws 91A is engaged with the corresponding engaging hole 92A.

By this operation, as shown in FIG. 15B, the protective member 80A formed by engaging the upper member 91 and the lower member 92 is engaged with the sheath 76 of the applicator 30A in a state of completely covering the connecting portion between the operation wire 62 and the clip 10. At this time, the protective member 80A is arranged in a state where the proximal end abuts against the sheath 76 and the distal end abuts against the holding tube 31 of the clip 10. Further, the first arm 12 and the second arm 13 of the arm member 11 of the clip 10 are in an open state in which they are separated from each other.

As shown in FIGS. 15A to 15C, the protective member 80A according to the present embodiment can cover the connecting portion between the operating wire 62 and the connecting rod 3 between the clip 10 and the applicator 30A in a state where the clip 10 is loaded to the applicator 30A. In the present embodiment, as shown in FIG. 15C, by attaching the protective member 80A to the distal end of the sheath 76, the region from the distal end of the protective member 80A to the distal end of the sheath 76 can be regarded as the first sheath according to the first embodiment described above. Further, in the present embodiment, the region from the distal end of the sheath 76 to the distal end surface 101c of the slit 101b of the operating portion 100 can be regarded as the second sheath according to the first embodiment described above. Therefore, in the present embodiment, in a state where the protective member 80A is attached to the support portion 76a on the distal end side of the sheath 76 of the applicator 30A, the region from the distal end of the protective member 80A to the distal end surface 101c of the slit 101b of the operating portion 100 can be regarded as a sheath including the first sheath and the second sheath according to the first embodiment described above. In the following description, the protective member 80A is defined as the first sheath, the sheath 76 of the applicator 30A is defined as the second sheath, and the region including the protective member 80A and the sheath 76 of the applicator 30A can be defined as the sheath 77.

Therefore, in the present embodiment, by attaching the protective member 80A to the support portion 76a on the distal end side of the sheath 76 of the applicator 30A, the length L of the sheath 77 can be adjusted from the second length L2 to the first length L1 which is larger than the second length L2. Further, for example, when the treatment of the target tissue is completed using the medical device 2 according to the present embodiment, when the protective member 80A is removed, the length L of the sheath 77 can be adjusted from the first length L1 to a smaller second length L2.

The operator can ligate the target tissue by using the medical device 2 according to the present embodiment in the same manner as in the first embodiment described above. Since the protective member 80A is not removed in the procedure for ligating the target tissue, the connecting portion between the hook 62a of the operation wire 62 and the connecting rod 3 does not move to a position protruding from the protective member 80A. In other words, according to the medical device 2 of the present embodiment, in the procedure for ligating the target tissue, the state in which the length L of the sheath 77 is the first length L1 is maintained, and the connecting portion between the hook 62a and the connecting rod 3 is always in an accommodation position of being accommodated in the protective member (first sheath) 80A.

According to the medical device 2 of the present embodiment, as in the first embodiment described above, both the reloadable clip 10 and the re-grasping operation of the target tissue T can be compatible with respect to the applicator 30A.

### Third Embodiment

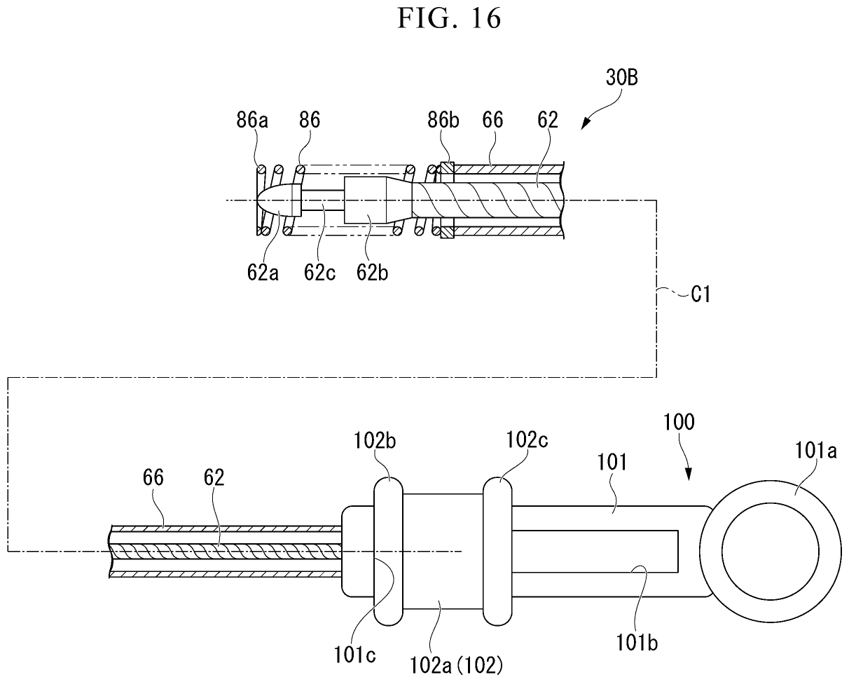

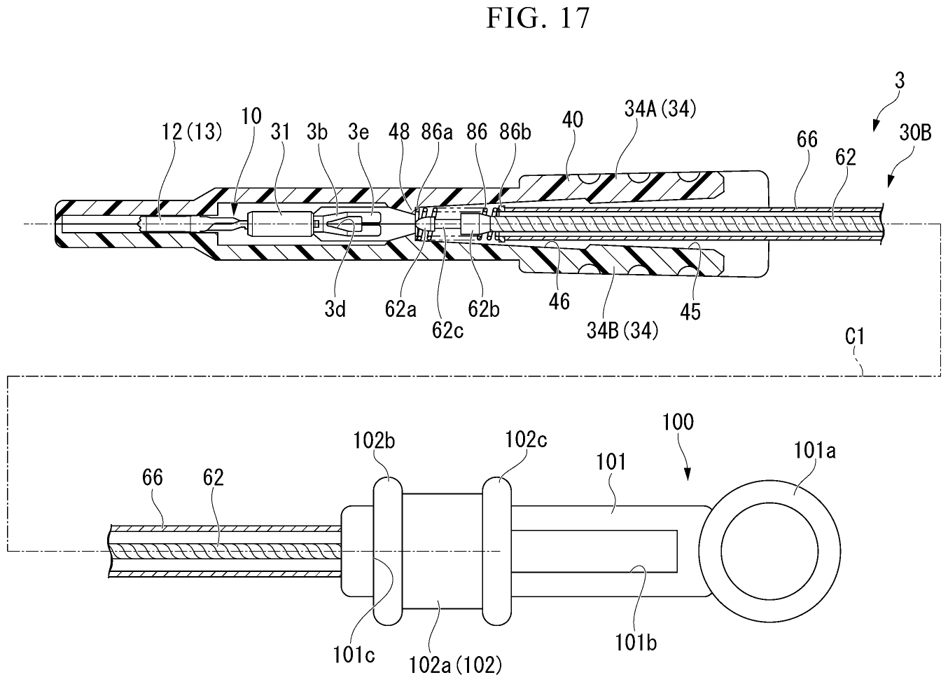

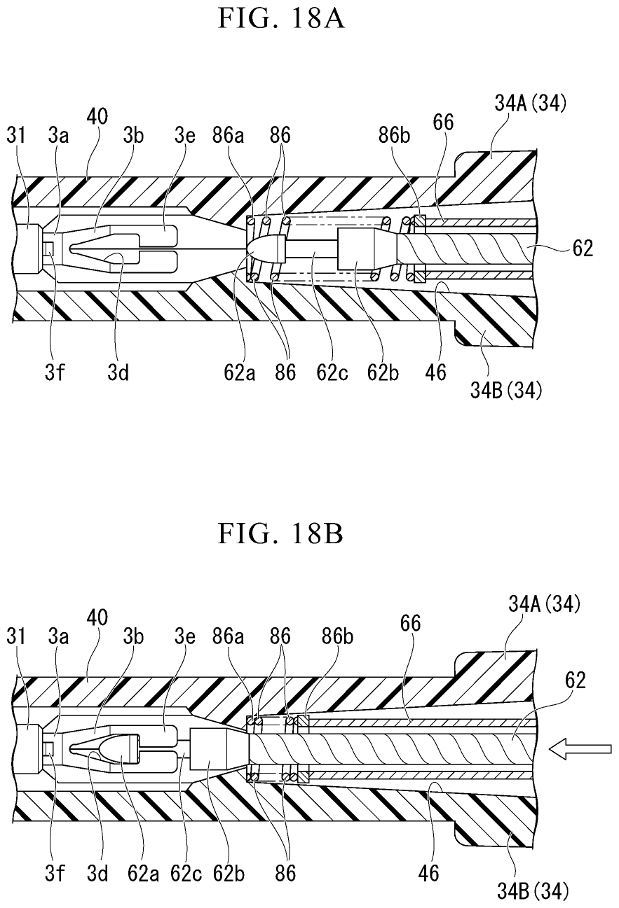

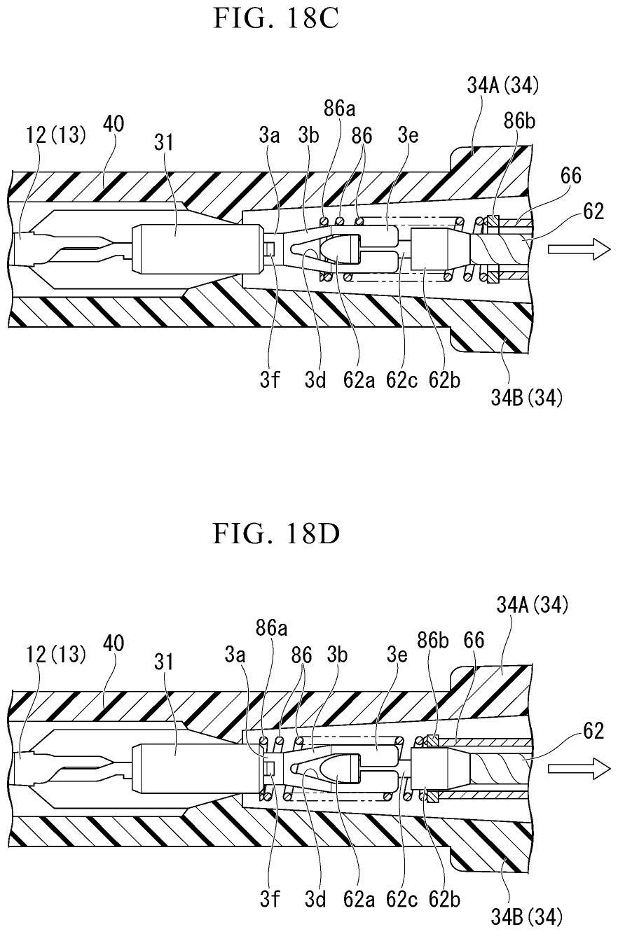

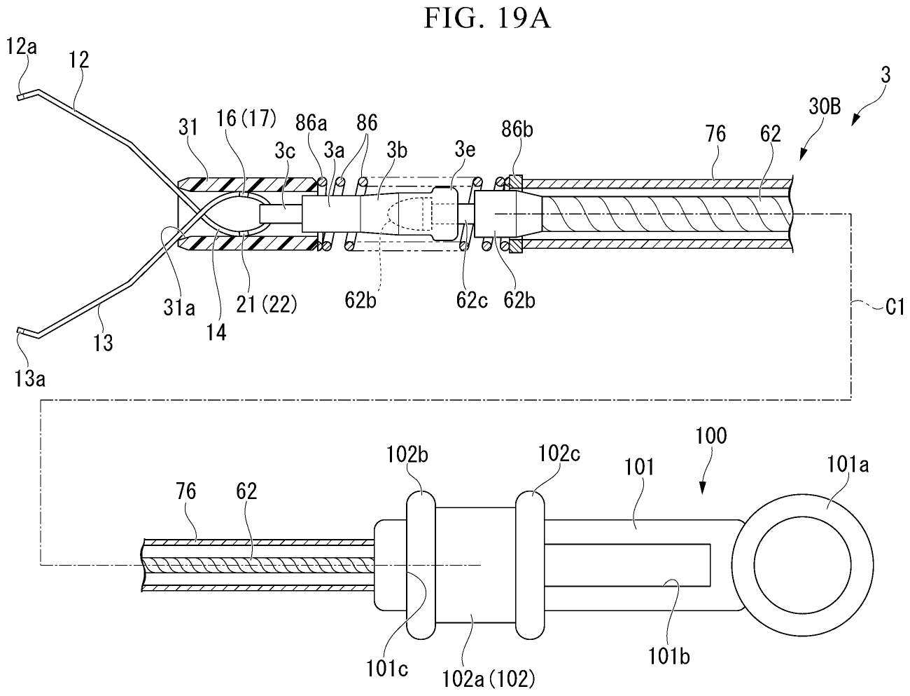

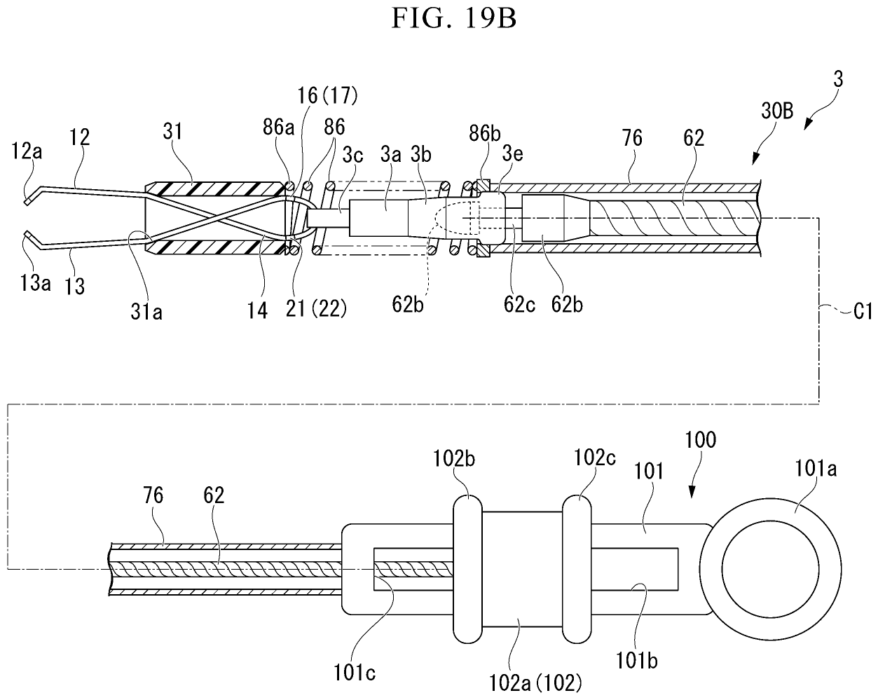

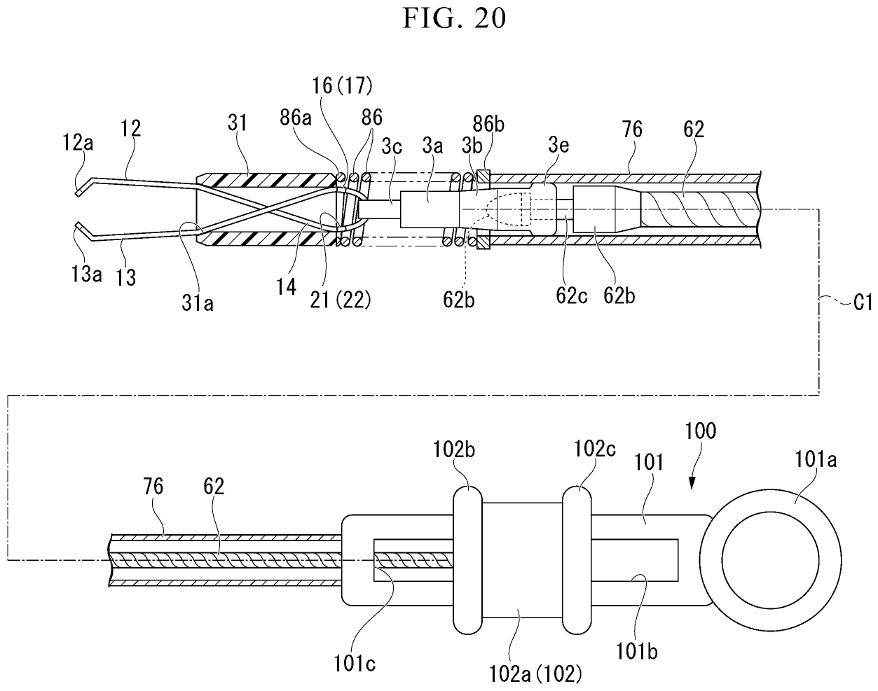

Next, the medical device according to the third embodiment of the present invention will be described with reference to FIGS. 16 to 20. FIG. 16 is a side view partial cross-sectional view showing the configuration of the applicator 30B according to the present embodiment. FIGS. 17 to 18D are views showing an operation of loading the clip 10 according to the present embodiment to the applicator 30B. FIGS. 19A to 20 are views showing an operation of treating the target tissue T using the medical device 3 according to the present embodiment.

Hereinafter, the same configuration as the medical device 1 according to the first embodiment will be described with the same reference numerals, the description thereof will be omitted, and the points different from those of the above-described embodiment will be mainly described.

As shown in FIG. 16, the applicator 30B of the medical device 3 according to the present embodiment is configured such that a spring (elastic member) 86 is attached to the distal end portion of the sheath 66 by a fixing member 86b. In the present embodiment, the spring 86 has an inner diameter such that the hook 62a and the connecting rod 3 are not disengaged from each other. More specifically, the spring 86 according to the present embodiment is formed to have an inner diameter such that the connecting rod 3 cannot rotate with respect to the hook 62a when the hook 82a and the connecting rod 3 are engaged with each other.

In the present embodiment, the spring 86 can be attached to the distal end of the sheath 66 by various known methods such as welding and adhesion. In the present embodiment, the region from the proximal end of the fixing member 86b to the distal end surface 101c of the slit 101b of the operating portion 100 is defined as the sheath 66.

In the present embodiment, by appropriately setting the natural length of the spring 86, as shown in FIG. 16, in a natural state in which an external force does not act on the spring 86, when the operator moves the slider 102 of the operating portion 100 to the position closest to the distal end side until it comes into contact with the distal end surface 101c of the slit 101b, the hook 62a provided at the distal end of the operation wire 62 is located closer to the proximal end than the distal end 86a of the spring 86.

In the present embodiment, the region where the spring 86 (including the fixing member 86b) is arranged is defined as a first sheath, the sheath 66 is defined as a second sheath, and the region including the first sheath and the second sheath is defined as a sheath 87. That is, as shown in FIG. 16, it can be said that the length L of the sheath 87 according to the present embodiment is the first length L1. In this state, the hook 62a is in the accommodation position accommodated in the first sheath.

When loading the clip 10 according to the present embodiment to the applicator 30B, as shown in FIG. 17, the operator moves the slider 102 to the position closest to the distal end surface 101c of the slit 101b until it comes into contact with the distal end surface 101c, and then inserts the applicator 30B into the clip cartridge 40. At this time, as shown in FIG. 18A, the distal end 86a of the spring 86 may come into contact with the step portion 48 of the clip cartridge 40 without pressing. That is, as shown in FIG. 18A, the length of the spring 86 is its own natural length.

After that, the operator does not operate the slider 102, but grasps the main body 101 and pushes the entire operating portion 100 toward the distal end side. At this time, as shown in FIG. 18B, the spring 86 receives the pressing force by the operator and presses against the stepped portion 48 of the clip cartridge 40. As a result, the length in the longitudinal axis direction becomes shorter than the natural length while the spring 86 is compressed. On the other hand, the hook 62a provided at the distal end of the operation wire 62 moves to the distal end side together with the operation wire 62.

In this process, the spring 86 is pressed and the length in the longitudinal axis direction becomes shorter than the natural length. Therefore, the length L of the sheath 87 according to the present embodiment is adjusted from the first length L1 to a smaller second length L2. As a result, the hook 62a provided on the distal end side of the operation wire 62 is moved from the accommodation position accommodated in the spring 86 to the protruding position protruding from the distal end 86a of the spring 86.

As a result, as shown in FIG. 18B, when the operator pushes the operating portion 100 toward the distal end side, the hook 62a is engaged with the notch portion 3d of the connecting rod 3. As a result, the clip 10 is connected to the operation wire 62 and loaded to the applicator 30B.

After confirming that the hook 62a is engaged with the notch portion 3d of the connecting rod 3 through the clip cartridge 40, the operator releases the pressing force acting on the operating portion 100. At this time, the elastic restoring force of the spring 86 acts on the sheath 66 and the operating portion 100 to move the spring 86 toward the proximal end side. As a result, the hook 62a and the clip 10 connected to the hook 62a move to the proximal end side. As described above, when the spring 86 has a natural length, the hook 62a is located closer to the proximal end side than the distal end 86a of the spring 86. Therefore, when the pressing force on the operating portion 100 by the operator is released and the spring 86 is restored to its natural length, as shown in FIG. 18C, the connecting portion of the hook 62a and the connecting rod 3 is located closer to the proximal end side than the distal end 86a of the spring 86, that is, is moved from the protruding position to the accommodation position.

FIG. 18C shows, as an example, an embodiment in which the distal end 86a of the spring 86 is not in contact with the holding tube 31, but the present invention is not limited thereto. For example, as shown in FIG. 18D, the distal end 86a of the spring 86 may be in contact with the holding tube 31. In either case shown in FIGS. 18C and 18D, since the connecting portion between the hook 62a and the connecting rod 3 is arranged inside the spring 86, the connection between the hook 62a and the connecting rod 3 is maintained.

After that, the operator removes the clip 10 loaded to the applicator 30B from the clip cartridge 40 together with the applicator 30B, and the operation of loading the clip 10 to the applicator 30 is completed.

The operator can ligate the target tissue by using the medical device 3 according to the present embodiment in the same manner as in the first embodiment described above. FIGS. 19A and 19B show an example of a procedure for ligating a target tissue using the medical device 3 according to the present embodiment.

As shown in FIG. 19A, in a state where the clip 10 in the medical device 3 according to the present embodiment is loaded to the applicator 30B, it is in an open state in which the holding tube 31 is in contact with the distal end 86a of the spring 86 and the first arm 12 and the second arm 13 of the arm member 11 of the clip 10 are separated from each other. In this state, when the operator pulls the operation wire 62 toward the proximal end side, the holding tube 31 presses the spring 86. In the present embodiment, since the initial tension of the spring 86 is set to be equal to or higher than the pressing force of the holding tube 31 pressing the spring 86, it is possible to prevent the spring 86 from being compressed. That is, as shown in FIG. 19B, in the process in which the first arm 12 and the second arm 13 of the arm member 11 transition from the open state to the closed state by the operator pulling the operation wire 62 toward the proximal end side, the length of the spring 86 in the direction of the longitudinal axis C1 does not change. As a result, the connecting portion between the hook 62a of the operation wire 62 and the connecting rod 3 does not move to a position protruding from the distal end 86a of the spring 86. In other words, according to the medical device 3 of the present embodiment, in the procedure for ligating the target tissue, the state in which the length L of the sheath 77 is the first length L1 is maintained, and the connecting portion between the hook 62a and the connecting rod 3 is always in the accommodation position accommodated in the spring 86.

As shown in FIG. 20, in a state where the initial tension of the spring 86 according to the present embodiment is lower than the pressing force for pressing the spring 86, when the operator pulls the operation wire 62, the spring 86 is compressed by pressing the spring 86 while the holding tube 31 is in contact with the distal end 86a of the spring 86. In this process, the holding tube 31 comes into contact with the distal end 86a of the compressed spring 86 and is moved to the proximal end side, so that the connecting portion between the hook 62a and the connecting rod 3 is always in the accommodation position accommodated in the spring 86 or the sheath 66.

When the operator pushes the slider 102 (pushes the operation wire 62) while the spring 86 is compressed, the compression of the spring 86 is released and the spring 86 returns to the original length, so that even when the slider 102 advances to the maximum, the connecting portion between the hook 62a and the connecting rod 3 is always in the accommodation position accommodated in the spring 86.

According to the medical device 3 of the present embodiment, as in each of the above-described embodiments, both the reloadability of the clip 10 and the re-grasping operation of the target tissue T can be achieved for the applicator 30B. Further, since the operation of loading the clip 10 to the applicator 30B is only the operation of pushing the operating portion 100 toward the distal end side, the operation of the operator can be made easier than in each of the above-described embodiments.

In the present embodiment, the configuration in which the spring 86 is provided on the distal end side of the sheath 66 has been described as an example, but the present invention is not limited thereto. For example, a configuration in which the spring 86 is provided at the intermediate portion or the proximal end portion of the sheath 66 has the same effect. In other words, in the present embodiment, when the region of the spring 86 is defined as the first sheath and the sheath 66 is defined as the second sheath, if at least one part of the first sheath provided on the distal end side and the second sheath located on the proximal end side of the first sheath is configured by the spring 86, it can be said that it has the same effect as the medical device 3 according to the present embodiment.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

According to each of the above-described embodiments of the present disclosure, it is possible to provide a medical device and an applicator capable of achieving both reloadability of the treatment tool and re-grasping operation of the target tissue by the treatment tool. Further, in this medical device, it is also possible to provide a method of suitably loading the treatment tool to the medical device by engaging the treatment tool with the applicator.

What is claimed is:

1. A medical device comprising:
a clip;
a wire configured to operate the clip;
a link configured to connect to and detach from the clip and the wire;
a sheath with an inner diameter configured to accommodate the link;
a handle connected to the sheath;
a slider provided on the handle and configured to move relative to the handle, the slider being connected to the wire, wherein:
the sheath includes:
a first sheath having a length such that the link protrudes from the first sheath when the slider is advanced with respect to the handle to a maximum extent; and
a second sheath being connected to the first sheath, the second sheath having a length such that the link is provided within the sheath when the slider is advanced relative to the handle to a maximum extent.

2. The medical device according to claim 1, wherein:
the clip includes a first link configured to engage with the link,
the link is configured to engage with the first link at a protruding position protruding from the sheath, and the sheath is configured to restrict disengagement with the first link at an accommodation position,
the sheath is configured to change between a first length, which is a length of the sheath when the link is placed in the accommodation position when the slider is maximally advanced with respect to the handle, and a second length, which is a length of the sheath when the link is placed in the protruding position when the slider is maximally advanced with respect to the handle.

3. The medical device according to claim 2, wherein, in a state where the first link and the second link are engaged and the length of the sheath is the first length, when the slider is maximally advanced, a connecting portion between the first link and the link are placed in the sheath.

4. The medical device according to claim 2, further comprising:
a first arm and a second arm provided on the clip; and
a holding tube provided in the clip and formed in a tubular shape, the holding tube being configured so that at least one of the first arm and the second arm can be inserted,
wherein the first link is arranged so as to protrude from the holding tube.

5. The medical device according to claim 4, wherein, in a state where the first link and the link are engaged and the length of the sheath is the first length, when the slider is maximally advanced, the holding tube and the sheath are in contact.

6. The medical device according to claim 1, further comprising:
a connecting member provided on the sheath, the connecting member connecting the first sheath and the second sheath and capable of moving the first sheath to a side of the second sheath with respect to the second sheath; and
a restricting part provided on the sheath and restricting movement of the first sheath with respect to the second sheath in a state where the sheath is a first length, which is a length of the sheath when the link is placed in the accommodation position when the slider is maximally advanced with respect to the handle.

7. The medical device according to claim 6, wherein
the restricting part restricts a movement of the first sheath with respect to the second sheath by a first force, and
the medical device further comprises an arm provided on the clip, the arm being configured to be closed by pulling the wire with a second force smaller than the first force.

8. The medical device according to claim 6, wherein at least one of the first sheath or the second sheath is connected to the connecting member in a state of being in a cavity formed in the connecting member.

9. The medical device according to claim 1, wherein
at least a part of either the first sheath or the second sheath is configured by a spring,
the sheath is configured to change between a first length, which is a length of the sheath when the link is accommodated in the sheath due to elastic deformation of the spring, and a second length, which is a length of the sheath when the link protrudes from the sheath.

10. The medical device according to claim 1, wherein, by transitioning between a form in which the first sheath and the second sheath are attached and a form in which the first sheath and the second sheath are separated, the sheath is configured to change between a first length, which is a length of the sheath when the link is accommodated in the sheath due to elastic deformation of a spring, and a second length, which is a length of the sheath when the link protrudes from the sheath.

11. An applicator configured to be connectable to a clip, the applicator comprising:
a link configured to connect to the clip;
a wire connected to the link;
a sheath that accommodates the link;
a handle connected to the sheath;
a slider provided on the handle and configured to move relative to the handle, the slider being connected to the wire;
the sheath including a first sheath and a second sheath, the first sheath having a length that allows the link to protrude from the first sheath when the slider is maximally advanced with respect to the handle; and
the second sheath being connected to the first sheath, the second sheath having a length that accommodates the link in the sheath when the slider is maximally advanced with respect to the handle.

12. The applicator according to claim 11, further comprising:
a connecting member provided on the sheath, the connecting member connecting the first sheath and the second sheath and being configured to move the first sheath to a side of the second sheath with respect to the second sheath; and
a restricting part provided on the sheath and restricting movement of the first sheath with respect to the second sheath when the sheath is provided at a first length.

13. The applicator according to claim 12, wherein
the restricting part restricts the movement of the first sheath with respect to the second sheath by a first force, and
the first force is larger than a second force, the second force being a traction force of a wire required when an arm provided in the clip closes.

14. The applicator according to claim 12, wherein
at least a part of either the first sheath or the second sheath includes a spring,
the sheath is configured to change between a first length, which is a length of the sheath when the link is accommodated in the sheath due to elastic deformation of the spring, and a second length, which is a length of the sheath when the link protrudes from the sheath.

15. The applicator according to claim 12, wherein, by transitioning between a first configuration where the first sheath and the second sheath are attached and a second configuration where the first sheath and the second sheath are not attached, the sheath is configured to change between a first length, which is a length of the sheath when the link is accommodated in the sheath due to elastic deformation of a spring, and a second length, which is a length of the sheath when the link protrudes from the sheath.

16. A loading method of a clip, comprising:
changing a length of a sheath of an applicator from a first length being is a length of the sheath that accommodates a link, to a second length, being a length that allows the link to protrude from the sheath; and
engaging the link with the clip when the sheath is at the second length.

17. The loading method according to claim 16, further comprising:
changing the length of the sheath from the first length to the second length by elastically deforming a spring forming a part of the sheath.

18. The loading method according to claim 17, further comprising:
deforming the spring elastically by pressing the sheath against a cartridge containing the clip.
